(19) United States
(12) Patent Application Publication
(10) Pub. No.: US 2026/0266821 A1
(43) Pub. Date: Sep. 10, 2026

(54) METHODS AND COMPOSITIONS FOR DETECTING INFLUENZA ANTIBODIES

(71) Applicant: Meso Scale Technologies, LLC., Rockville, MD (US)

(72) Inventors: George Sigal, Rockville, MD (US); Laura Ahlers, Rockville, MD (US); Nicolas Sammons, Rockville, MD (US); Brian Lane, Rockville, MD (US); Leonid Dzantiev, Rockville, MD (US); Yeming Wang, Rockville, MD (US)

(21) Appl. No.: 19/558,010

(22) Filed: Mar. 5, 2026

Related U.S. Application Data
(60) Provisional application No. 63/767,453, filed on Mar. 5, 2025.

(51) Int. Cl. G01N 33/569; G01N 33/53; G01N 33/543; G01N 33/564

(52) U.S. Cl. CPC G01N 33/56983; G01N 33/5306; G01N 33/54393; G01N 33/564; G01N 2333/11; G01N 2458/00; G01N 2800/26

(57) ABSTRACT

Methods and compositions that the detection and quantification of antibodies that bind to one or more influenza antigens in a biological sample are provided. The detection is based on the formation of a bridging complex, where a bivalent antibody binds to two antigens, a first antigen linked to a detectable label, and a second antigen linked to a binding moiety for immobilizing the complex on a solid substrate via a binding partner. The methods further include an unlabeled influenza antigen, such as H1N1 hemagglutinin, which acts as a blocking agent to neutralize cross-reactive antibodies (e.g., anti-H1N1 antibodies). The disclosed methods minimize false positives, thereby facilitate increased accuracy in detecting anti-H5N1 antibodies. Also provided are methods for detecting influenza in a subject, diagnosing infection, and guiding treatment decisions.

### CROSS-REFERENCE TO RELATED APPLICATIONS

U.S. Provisional Application No. 63/767,453, filed Mar. 5, 2025, which is specifically incorporated herein by reference in its entirety.

### STATEMENT REGARDING FEDERALLY SPONSORED RESEARCH

This invention was made with government support under grant number AI176136, awarded by the National Institutes of Health. The government has certain rights in the invention.

### REFERENCE TO SEQUENCE LISTING

The Sequence Listing XML submitted as a file named “MSD_AT0800US.xml”, created on Mar. 5, 2026, and having a file size of 23,163 bytes is hereby incorporated by reference pursuant to 37 C.F.R. § 1.834(c)(1).

### FIELD OF THE INVENTION

The disclosed invention is generally related to compositions, methods, and kits for detecting H5 influenza.

### BACKGROUND OF THE INVENTION

Clade 2.3.4.4b H5N1 influenza is a highly pathogenic avian influenza virus that can spread widely among bird populations and has demonstrated the ability to infect a variety of mammalian species, including dairy cattle. Sporadic zoonotic transmissions to humans have also been reported. Accurate assessment of the extent of human H5N1 transmission is important for understanding zoonotic risk and informing public health interventions.

Currently, serological detection of anti-H5 antibodies is being used to screen for evidence of prior H5N1 infection, particularly in high-risk populations such as agricultural workers who are frequently exposed to infected animals. However, antibody-based H5 surveillance faces a significant challenge due to the high degree of cross-reactivity between antibodies targeting the hemagglutinin (HA) protein of endemic H1N1 influenza and the related HA of the H5N1 virus, both belonging to Type A Group 1 influenza viruses. This cross-reactivity increases the risk of false positive results, particularly in populations with frequent H1N1 exposure, and complicates efforts to reliably distinguish true H5N1 seroconversion from background immunity to seasonal influenza.

Thus, there is a need for more effective antibody detection methods with higher specificity to distinguish true H5N1 infections from cross-reactive responses to seasonal influenza.

It is an object of the present invention to provide methods for detecting H5N1 antibodies with improved specificity by reducing cross-reactivity with H1N1 antibodies.

It is a further object of the present invention to provide compositions that can be used in detecting H5N1 antibodies with improved specificity by reducing cross-reactivity with H1N1 antibodies.

It is a further object of the present invention to provide kits that can be used in detecting H5N1 antibodies with improved specificity by reducing cross-reactivity with H1N1 antibodies.

### SUMMARY OF THE INVENTION

The disclosed serological methods facilitate the detection and quantification of antibodies that bind to one or more influenza antigens in a biological sample. The methods rely on the formation of a bridging complex, where a bivalent antibody in the sample binds to two antigens, one linked to a detectable label and the other linked to a binding partner that facilitates immobilization on a solid support.

In some forms, the antibody is an anti-H5N1 antibody, and the antigens are H5N1 antigens, such as hemagglutinin (HA). This dual-antigen binding mechanism increases specificity by only allowing antibodies capable of recognizing both antigens to contribute to the assay signal, thereby reducing cross-reactivity and increasing specificity.

The methods further incorporate an unlabeled influenza antigen, such as H1N1 hemagglutinin, which serves as a blocking agent to neutralize cross-reactive antibodies, such as anti-H1N1 antibodies, that may be present in the sample. By preventing these antibodies from binding to the H5N1 antigens used in the assay, this H1 blocking step reduces cross-reactivity-driven false positives, thereby facilitating more accurate detection of true anti-H5N1 antibodies.

In one exemplary form, the method measures antibodies that bind to influenza hemagglutinin (HA) of the H5 subtype in a sample; the steps including:
a. forming a bridging complex containing (i) an antibody from the sample having a first and a second binding site for H5 HA; (ii) a first modified H5 HA antigen bound to the first binding site; and (iii) a second modified H5 HA antigen bound to the second binding site; and
b. measuring the formed bridging complex.

In some forms, a “modified antigen” (e.g., as in modified H5 HA antigen) refers generally to an antigen that has been altered in a manner that facilitates detection, capture, immobilization, or measurement of antigen-antibody complexes formed during the method. For example, the first H5 HA antigen and the second H5 HA antigen can be modified by attachment or association with a detectable label and/or binding moiety. Generally, the modification includes addition of a detectable label, a tag, or other functional group to the antigen, allowing the antigen or the resulting antigen-antibody complex to be detected or monitored during performance of the method, while otherwise retaining the antigenic properties of the antigen. In some forms, the first H5 HA antigen is modified by conjugation to a detectable label that allows detection of the bridging complex. In some forms, the second H5 HA antigen is modified by conjugation to a binding moiety (e.g., biotin) that binds a corresponding binding partner (e.g., streptavidin), for example on a solid support. Such modifications can be achieved through chemical, biochemical, or affinity-based attachment of a label, tag, or other binding functionality that facilitates detection, capture, or measurement of the bridging complex.

Methods for detecting an influenza virus in a subject are also provided. Also provided are methods for diagnosing an influenza infection in a subject, and treating the subject.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings illustrate several embodiments of the disclosed method and compositions and together with the description, serve to explain the principles of the disclosed method and compositions.

FIG. 1 is a schematic representation of the binding interactions needed to generate a signal in an exemplary bridging serology format. The head domain of an H5 HA is used as the antigen for this exemplary H5 assay.

FIG. 2 is a graph showing the levels of anti-H5 antibody activity of negative (circle) and positive pooled H5 vaccine study (star) samples measured in the absence (left panel) or presence (right panel) of free H1 HA to remove H1 activity. The vaccine study samples are labeled to indicate the vaccine strain (A/Vietnam/1203/04 or A/Indonesia/5/2005) and the HAI titer reported by BEI against the vaccine strain.

### DETAILED DESCRIPTION OF THE INVENTION

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### I. Definitions

The term “assay” refers to an in vitro procedure for analyzing a sample to determine the presence, absence, or quantity of one or more analytes of interest.

The terms “control” and “calibration” as used in connection with analytes, are used interchangeably to refer to analytes used as internal standards.

The term “analyte” refers to a chemical substance of interest that is a potential constituent of a biological sample and is to be analyzed by an assay.

The term “immobilized” refers to chemical or physical fixation of an agent or particle to a location on or in a substrate, such as a membrane. For example, capture agents may be chemically conjugated to a membrane, and particles coated with capture agents may be physically trapped within a membrane.

The term “biological sample” refers to a tissue (e.g., tissue biopsy), organ, cell, cell lysate, or body fluid from a subject. Non-limiting examples of body fluids include blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid.

The term “specifically binds” or “selectively binds” refers to a binding reaction which is determinative of the presence of the analyte in a heterogeneous population. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### II. Influenza Antigens and Bridging Immunoassay

Existing methods for detecting antibodies against H5N1 influenza virus face significant limitations in accurately distinguishing true H5N1-specific antibodies from cross-reactive antibodies generated by exposure to related influenza subtypes, such seasonal H1N1 influenza viruses. Traditional hemagglutination inhibition (HI) assays and microneutralization (MN) assays are widely used for serological surveillance of influenza infections, including H5N1. These assays are labor-intensive to perform and require the use of live viruses. In the case of H5N1, this requirement significantly restricts the use of such assays to laboratories equipped with Biosafety Level 3 (BSL-3) facilities. These assays often lack sufficient specificity to reliably differentiate between antibodies raised against H5N1 hemagglutinin (HA) and antibodies that cross-react due to prior exposure to Group 1 influenza A viruses, such as HINI, which shares conserved structural features with H5N1 HA. This cross-reactivity increases the risk of false positive results, particularly in populations with high levels of seasonal H1N1 exposure or vaccination. Other formats, such as indirect serology assays often performed using enzyme-linked immunosorbent assays (ELISA) or lateral flow devices eliminate the need for live virus. However, these formats still face challenges related to limited specificity.

The need for improved specificity is important for example, in serological surveillance programs that aim to assess the extent of human exposure to zoonotic H5N1 viruses, including individuals working in close contact with poultry, livestock, or other animals known to carry H5N1. False positives caused by cross-reactive H1N1 antibodies can artificially inflate estimates of human H5N1 exposure, leading to misleading epidemiological data and complicating risk assessments.

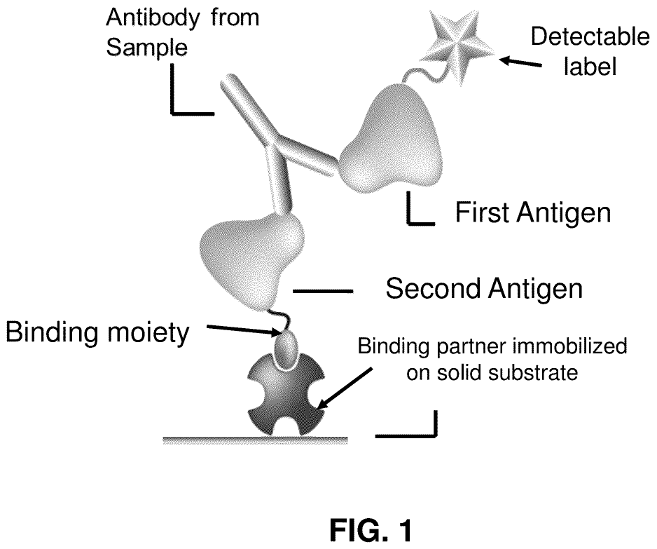

Bridging immunoassays are disclosed herein. Generally, the bridging immunoassays detect target antibodies by forming a bridging complex. The bridging complex includes (i) a target antibody in the sample (e.g., anti-H5N1 antibody), having a first and a second binding site for an H5N1 antigen (e.g., H5N1 HA), (ii) a first modified H5N1 antigen (also referred to herein as “first antigen”) bound to the first binding site of the antibody, and (iii) a second modified H5N1 antigen (also referred to herein as “second antigen”) bound to the second binding site of the target antibody. In some forms, the bridging immunoassay detects antibodies by forming a bridging complex, where each arm of a bivalent antibody binds to a first antigen, a first antigen labeled with a detectable tag and the second antigen linked to a binding moiety for immobilization. (see, e.g. FIG. 1), respectively. In other forms, the bridging immunoassay can be performed in solution, without the need for immobilization. The dual-antigen binding increases specificity by allowing only antibodies capable of binding both the first and second antigens to contribute to the signal. In some forms, the assay also includes a blocking antigen, which binds cross-reactive antibodies, preventing them from participating in bridging complex formation, thereby increasing the specificity of antibody detection in biological samples.

In some forms, the antigens and/or blockers are monomeric HA1 fragments. In some forms, to keep the antigen(s) and/or blocker(s) monomeric, this fragment omits most of the stem domain (also referred to as stalk domain). In some forms, the full HA ectodomain (HA1 globular head+HA2 stalk domain) is used. The HA1 head domain is the region of influenza HA trimer that binds host-cell surface receptors, whereas the HA2 stalk domain is the portion of the influenza HA trimer that catalyzes virus envelope fusion with the host cell membrane. In some forms, the stalk domain is inactivated, e.g., by mutation. In some forms, constructs can include the full stalk domain or other trimerization domains, such as those from T4 bacteriophage, although the may lead to increased background signal. Exemplary antigen and blocker amino acid sequences are provided below.

As demonstrated in the non-limiting Examples, an exemplary version of the assay was applied to human serum samples, including samples from individuals with known exposure to H5N1 and archived samples from presumably H5-naïve individuals with prior H1N1 exposure. The assay effectively distinguished true H5N1 seroconversion from background cross-reactivity. The inclusion of the H1 blocking antigen significantly increased the positive predictive value (PPV) of the assay compared to traditional methods that lack cross-reactive blocking.

The term “antibody” refers to intact immunoglobulin molecules, fragments or polymers of immunoglobulin molecules, single chain immunoglobulin molecules, human or humanized versions of immunoglobulin molecules, and recombinant immunoglobulin molecules, as long as they are chosen for their ability to bind an analyte.

The antigens themselves, as well as the methods for detecting them, and their uses for diagnosing infectious diseases e.g., H5N1, and guiding subsequent treatment, are provided.

### A. Influenza Viruses to be Detected

The influenza virus to be detected using the disclosed methods is an Influenza A virus. For example, the influenza virus to be detected can be an Influenza A virus subtype H5N1 (A/H5N1).

A/H5N1 is a diverse and evolving family of highly pathogenic avian influenza viruses that have circulated in wild birds, domestic poultry, mammals, and occasionally humans since it was first identified in 1996. Over time, genetic changes have led to the emergence of numerous strains, grouped into clades, each adapted to different hosts and environments. In the case of influenza viruses like H5N1, a clade refers to a genetically distinct group of related virus strains that evolved from a common ancestral strain. Clades are grouped based on similarities and differences in the virus's genetic sequence, e.g., in genes such as hemagglutinin (HA). For example, Clade 0 includes the original A/Goose/Guangdong/1/1996 strain, while Clade 1 encompasses strains responsible for human infections in Vietnam and Thailand in the early 2000s. Other clades, such as 2.1 in Indonesia and 2.2 in Europe and Africa, reflect how the H5N1 virus spread through different regions via wild bird migration and trade. In recent years, the 2.3.4.4b clade is the dominant global lineage, responsible for the ongoing global panzootic, a widespread outbreak in birds across multiple continents, along with spillover infections into mammals, including sea lions, foxes, and dairy cattle, as well as sporadic human cases. Each named strain, such as A/Vietnam/1203/2004 or A/Chile/2022, provides a snapshot of the virus at a particular time and place. More detailed reviews on the emergence of H5N1 strains are further described for example, in Charostad, et al., Travel Medicine and Infectious Disease, vol. 55, article 102638, (2023); Peacock, et al., Nature, vol. 637, pages 304-313 (2025); Webby and Uyeki, Journal of Infectious Diseases, 230(3): 533-542 (2024), the contents of which are incorporated herein by reference in their entireties.

The disclosed methods can be used to detect any strain of the A/H5N1. For example, the methods can be used to detect any one or more of the following A/H5N1 strains: A/Goose/Guangdong/1/1996, A/Hong Kong/156/1997, A/Vietnam/1203/2004, A/Indonesia/5/2005, A/Turkey/1/2005, A/Egypt/321/2007, A/Bangladesh/207095/2011, A/gyrfalcon/Washington/41088-6/2014, A/Guangdong/Thoroughbred horse/2017, A/Astrakhan/3212/2020, A/Ghana/AVL-763_21VIR7050-39/2021, A/Canada/ON-FAV-001/2022, A/France/2022, A/USA/ID/2023, A/Peru/2023. The methods can also be used to detect emerging A/H5N1 strains descending from a previously existing or currently existing A/H5N1 strain.

The genome of the H5N1 genome contains a negative single-stranded RNA molecule, spanning approximately 13.5 kilobases in length. The genome is segmented into eight segments, each encoding specific proteins that play critical roles in the viral lifecycle. These proteins include polymerase basic 1 (PB1, 757 amino acids), polymerase basic 2 (PB2, 759 amino acids), polymerase acidic (PA, 716 amino acids), hemagglutinin (HA, 568 amino acids), nucleoprotein (NP, 498 amino acids), neuraminidase (NA, 499 amino acids), matrix protein 1 (M1, 252 amino acids), matrix protein 2 (M2, 97 amino acids), as well as nonstructural proteins NS1 (225 amino acids) and NS2 (121 amino acids). The HA and NA glycoproteins dominate the viral surface and exist in several different forms and have high mutation rate.

The genome viral assemblies for H5N1 are accessible on NCBI Virus database under the following assembly numbers: GCA_038784385.1, GCA_038784375.1, GCA_038783835.1, GCA_038783815.1, all of which are incorporated herein by reference in their entireties.

In some forms, the bridging immunoassay can be adapted to detect antibodies against other viral species, such as H1N1, while using blocking antigens derived from H5N1 to reduce cross-reactivity. In other forms, the bridging immunoassay can be adapted for other applications where cross-reactivity between related viral species presents a challenge, with blocking antigens selected to suppress unwanted signals from non-target antibodies.

### B. Forming a Bridging Complex

The methods include contacting a sample with one or more antigens under conditions suitable to promote the formation of a bridging complex. A bridging complex forms when each arm of a bivalent antibody in the sample binds to a separate antigen, physically linking the antigens together.

The formation of the bridging complex relies on the bivalent nature of the antibody, which contains two antigen-binding sites capable of engaging both the first and second antigens at the same time. This results in a ternary structure, where the antibody acts as a physical bridge connecting the labeled first antigen with the binding partner-linked second antigen. This configuration allows the entire complex to be immobilized, detected, or quantified based on a detectable label and provides a means to measure the presence of the antibody in the sample.

In exemplary forms, the bridging complex forms when a first antigen, linked to a first label (e.g., a first detectable label), binds to a first binding site on the antibody, while a second antigen, linked to a binding partner (e.g., a binding moiety or a second label), binds to a second binding site on the antibody. For example, the first antigen may bind to a paratope on one Fab arm of the antibody, while the second antigen binds to a paratope on the opposite Fab arm. In some forms, the first and second antigens can be identical or can contain different portions or variants of the same target antigen, allowing them to simultaneously bind both antigen-binding sites on the antibody.

The first and second antigens used to form the bridging complex are generally modified to facilitate detection and/or capture of the bridging complex formed with the antibody. Generally, the modification includes addition of a detectable label, a tag, or other functional group to the antigen, thereby allowing the antigen or the resulting antigen-antibody complex to be detected or monitored during performance of the method, while otherwise retaining the antigenic properties of the antigen. For example, the first and second antigens can be modified to include tags, linkers, or chemical moieties to facilitate conjugation to a detectable label or a solid-phase support. In some forms, the first antigen can be modified by attachment of a detectable label (e.g., a SULFO-Tag™) that allows measurement and detection of the bridging complex using a suitable detection platform. In some forms, the second antigen can be modified by attachment of a binding moiety (e.g., biotin) that allows the bridging complex to be immobilized through interaction with a corresponding binding partner (e.g., streptavidin), for example on a solid support. In some forms, both the first and second antigens are modified, such that each antigen includes a different modification. For example, one antigen (e.g., the first antigen) includes a detectable label and the other antigen (e.g., the second antigen) includes a binding partner/moiety. These modifications can be achieved through chemical conjugation, affinity-based interactions, or other labeling approaches that facilitate formation, detection, capture, and/or quantification of the bridging complex.

The formation of the bridging complex requires that the antibody possess sufficient affinity and specificity to bind both the first and second antigens, thereby providing higher specificity for antibodies capable of recognizing the antigen. This minimizes non-specific interactions and improves the discrimination of the target antibody from unrelated antibodies or background signal.

### 1. First and Second Antigens

The first and second antigens can include full-length proteins, protein fragments, peptides, recombinant proteins, or fusion proteins derived from A/H5N1 influenza virus. For example, the first and/or second antigen can include the full ectodomain of an A/H5N1 viral surface protein, such as hemagglutinin (HA). The HA serves as the primary glycoprotein on the surface of the H5N1 virus and is involved in the early stages of viral infection. HA is responsible for binding the virus to specific receptors on the surface of host cells, facilitating viral entry. Additionally, the HA protein plays a role in the liberation of the viral genome into the host cell's cytoplasm by inducing membrane fusion.

In some forms, the first and/or second antigen can include specific domains or regions of the HA protein, such as the head domain or receptor-binding domain (RBD). In other forms, the first and/or second antigen includes amino acids from the HA1 HA fragment. Exemplary amino acid sequences for A/H5N1 HA proteins that can be used as the first and/or second antigens are provided as SEQ ID NO:1, SEQ ID NO:2, SEQ ID NO:3, SEQ ID NO:7, SEQ ID NO:8, SEQ ID NO:9, or SEQ ID NO:10, which are described below. Some of the amino acid sequences include a signal peptide sequence and/or ectodomain (e.g., as annotated). Each of the disclosed sequences are expressed provided with and without the signal peptide sequence and/or with the ectodomain sequence or an antibody-binding portion thereof.
HA [Influenza A virus](strain A/Chicken/Hong Kong/220/1997 H5N1 genotype Gs/Gd)
UniProt ID: O89746⋅HEMA_I97A0; 568 amino acids

(SEQ ID NO: 1)
MEKIVLLLATVSLVKSDQICIGYHANNSTEQVDTIMEKNVTVTHA
QDILERTHNGKLCDLNGVKPLILRDCSVAGWLLGNPMCDEFINVP
EWSYIVEKASPANDLCYPGNENDYEELKHLLSRINHFEKIQIIPK
SSWSNHDASSGVSSACPYLGRSSFFRNVVWLIKKNSTYPTIKRSY
NNTNQEDLLVLWGIHHPNDAAEQTKLYQNPTTYISVGTSTLNQRL
VPEIATRPKVNGQSGRMEFFWTILKPNDAINFESNGNFIAPEYAY
KIVKKGDSTIMKSELEYGNCNTKCQTPMGAINSSMPFHNIHPLTI
GECPKYVKSNRLVLATGLRNTPQRERRRKKRGLFGAIAGFIEGGW
QGMVDGWYGYHHSNEQGSGYAADQESTQKAIDGVINKVNSIINKM
NTQFEAVGREFNNLERRIENLNKKMEDGELDVWTYNTELLVLMEN
ERTLDFHDSNVKNLYDKVRLQLRDNAKELGNGCFEFYHKCDNECM
ESVKNGTYDYPQYSEEARLNREEISGVKLESMGTYQILSIYSTVA
SSLALAIMVAGLSLWMCSNGSLQCRICI.

HA [Influenza A virus](strain A/Goose/Guangdong/1/1996 H5N1 genotype Gs/Gd)
(UniProt ID: Q9Q0U6⋅HEMA_I96A0, 568 amino acids)

(SEQ ID NO: 2)
MEKIVLLLAIVSLVKSDQICIGYHANNSTEQVDTIMEKNVTVTHA
QDILEKTHNGKLCDLNGVKPLILRDCSVAGWLLGNPMCDEFINVP
EWSYIVEKASPANDLCYPGDENDYEELKHLLSRTNHFEKIQIIPK
SSWSNHDASSGVSSACPYHGRSSFFRNVVWLIKKNSAYPTIKRSY
NNTNQEDLLVLWGIHHPNDAAEQTKLYQNPTTYISVGTSTLNQRL
VPEIATRPKVNGQSGRMEFFWTILKPNDAINFESNGNFIAPEYAY
KIVKKGDSAIMKSELEYGNCNTKCQTPMGAINSSMPFHNIHPLTI
GECPKYVKSNRLVLATGLRNTPQRERRRKKRGLFGAIAGFIEGGW
QGMVDGWYGYHHSNEQGSGYAADKESTQKAIDGVINKVNSIIDKM
NTQFEAVGREFNNLERRIENLNKQMEDGFLDVWTYNAELLVLMEN
ERTLDFHDSNVKNLYDKVRLQLRDNAKELGNGCFEFYHKCDNECM
ESVKNGTYDYPQYSEEARLNREEISGVKLESMGTYQILSIYSTVA
SSLALAIMVAGLSLWMCSNGSLQCRICI.

HA [Influenza A virus](A/bar-headed goose/Qinghai/14/2008(H5N1))
(NCBI Accession Number: ACL28277.1; 569 amino acids)

(SEQ ID NO: 3)
MEKIVLLLAIVSLVKSDQICIGYHANNSTEQVDTIMEKNVTVTHA
QDILEKTHNGKLCDLNGVKPLILRDCSVAGWLLGNPMCDEFTNVP
EWSYIVEKASPANDLCYPGDENDYEELKHLLSRINHFEKIQIIPK
SSWSNHDASSPVSSACPYHGRSSFERNVVWLIKKNSTYPTIKRSY
NNTNQEDLLVLWGIHHPNDAAEQTKLYQNPTTYISVGTSTLNQRL
VPEIATRPKVNGQSGRMEFFWTILKPIDAINFESNGNFIAPEYAY
KIVKERDSAIMKSELEYGNCNTKCQTPMGAINSSMPFHNIHPLTI
GECPKYVKSNRLVLATGLRNTPQRERRRKKRGLFGAIAGFIEGGW
QGMVDGWYGYHHSNEQGSGYAADKESTQKAIDGVINKVNSIIDKM
NTQFEAVGREFNNLERRIESLNKKMEDGELDVWTYNAELLVLMEN
ERTLDFHDSNVKNLYDKVRLQLRDNAKELGNGCFEFYHKCDNECM
ESVKNGTYDYPQYSEEARLNREEISGVKLESMGTYQILSIYSTVC
ELPSTGNHGSLVLSLWMSNGSLQCRICI.

HA [Influenza A/chicken/Ghana/AVL-763_21VIR7050-39/2021 (H5N1)]
H5 Clade: 2.3.4.4b (GISAID Accession Number: EPI2415855; 567 amino acids)
MENIVLLLAIVSLVKSDQICIGYHANNSTEQVDTIMEKNVTVTHAQDILEKTHNGKLCD LNGVKPLILKDCSVAGWLLGNPMCDEFIRVPEWSYIVERANPANDLCYPGSLNDYEELK HLLSRINHFEKILIIPKSSWPNHETSLGVSAACPYQGAPSFFRNVVWLIKKNDTYPTIK ISYNNTNREDLLILWGIHHSNNAEEQTNLYKNPTTYISVGTSTLNQRLVPKIATRSQVN GQRGRMDFFWTILKPDDAIHFESNGNFIAPEYAYKIVKKGDSTIMKSGVEYGHCNTKCQ TPVGAINSSMPFHNIHPLTIGECPKYVKSNKLVLATGLRNSPLREKRRKRGLFGAIAGF IEGGWQGMVDGWYGYHHSNEQGSGYAADKESTQKAIDGVTNKVNSIIDKMNTQFEAVGR EFNNLERRIENLNKKMEDGFLDVWTYNAELLVLMENERTLDFHDSNVKNLYNKVRLQLR DNAKELGNGCFEFYHKCDNECMESVRNGTYDYPQYSEEARLKREE ISgvklesigtyqi lsiystaasslalaimmaglslwmcsngslqcrici (SEQ ID NO:7). The uppercase bolded amino acids in SEQ ID NO:7 represents the signal peptide that is cleaved during expression. Uppercase and un-bolded amino acids in SEQ ID NO:7 represent the ectodomain. The lowercase and bolded amino acids in SEQ ID NO:7 represent the membrane portion of the HA protein.
HA [Influenza A/Texas/37/2024 (H5N1); H5 Clade: 2.3.4.4b]
GISAID Accession Number: EPI3171488; 567 amino acids
MENIVLLLAIVSLVKSDQICIGYHANNSTEQVDTIMEKNVTVTHAQDILEKTHNGKLCD LNGVKPLILKDCSVAGWLLGNPMCDEFIRVPEWSYIVERANPANDLCYPGSLNDYEELK HMLSRINHFEKIQIIPKSSWPNHETSLGVSAACPYQGAPSFFRNVVWLIKKNDAYPTIK ISYNNTNREDLLILWGIHHSNNAEEQTNLYKNPITYISVGTSTLNQRLAPKIATRSQVN GQRGRMDFFWTILKPDDAIHFESNGNFIAPEYAYKIVKKGDSTIMKSGVEYGHCNTKCQ TPVGAINSSMPFHNIHPLTIGECPKYVKSNKLVLATGLRNSPLREKRRKRGLFGAIAGF IEGGWQGMVDGWYGYHHSNEQGSGYAADKESTQKAIDGVTNKVNSIIDKMNTQFEAVGR EFNNLERRIENLNKKMEDGFLDVWTYNAELLVLMENERTLDFHDSNVKNLYDKVRLQLR DNAKELGNGCFEFYHKCDNECMESVRNGTYDYPQYSEEARLKREEISgvklesvgtyqi lsiystaasslalaimmaglslwmcsngslqcrici (SEQ ID NO:8). The uppercase bolded amino acids in SEQ ID NO:8 represents the signal peptide that is cleaved during expression. Uppercase and un-bolded amino acids in SEQ ID NO:8 represent the ectodomain. The lowercase and bolded amino acids in SEQ ID NO:8 represent the membrane portion of the HA protein.
HA [Influenza A/Cambodia/NPH230032/2023 (H5N1); H5 Clade: 2.3.2.1c]
GISAID Accession Number: EPI2419700; 567 amino acids
MKKIVLLFATISLVKSDHICIGYHANNSTEKVDTIMEKNVTVTQAQDILEKTHNGKLCD LNGVKPLILKDCSVAGWLLGNPLCDEFTNVPEWSYIVEKANPVNDLCYPGSFNDYEELK HLLSRINHFEKIQIIPKNSWSDHEASLGVSATCSYQGNSSFFRNVVWLIKKNNAYPTIK KDYNNTNREDLLILWGIHHPNDEAEQTRLYQNPTTYISIGTSTLNQRLVPRIATRPKIN GQSGRIDFFWTILKPNDAIHFESNGNFIAPEYAYKIIKKGDSTIMRSEVKYGNCNTRCQ TPIGAINSSMPFHNIHPLTIGECPKYVKSSKLVLATGLRNSPQKERRRKRGLFGAIAGF IEGGWQGMVDGWYGYHHSNEQGSGYAADKESTQPAIDGVTNKVNSIIDKMNTQFEAVGR EFNNLERRIENLNKKMEDGFLDVWTYNAELLVLMENERTLDFHDSNVRNLYDKVRLQLK DNAKELGNGCFEFYHKCNNECMESVRNGTYDYPQYSEEARLKREEISgvklesmgiyqi lsiystvasslvlaimmaglslwmcsngslqcrici (SEQ ID NO:9). The uppercase bolded amino acids in SEQ ID NO:9 represents the signal peptide that is cleaved during expression. Uppercase and un-bolded amino acids in SEQ ID NO:9 represent the ectodomain. The lowercase and bolded amino acids in SEQ ID NO:9 represent the membrane portion of the HA protein.
HA [Influenza A/Indonesia/5/2005 (H5N1); H5 Clade: 2.1.3.2]
GISAID Accession Number: EPI2419700
MEKIVLLLAIVSLVKSDQICIGYHANNSTEQVDTIMEKNVTVTHAQDILEKTHNGKLCD LDGVKPLILRDCSVAGWLLGNPMCDEFINVPEWSYIVEKANPTNDLCYPGSFNDYEELK HLLSRINHFEKIQIIPKSSWSDHEASSGVSSACPYLGSPSFFRNVVWLIKKNSTYPTIK KSYNNTNQEDLLVLWGIHHPNDAAEQTRLYQNPTTYISIGTSTLNQRLVPKIATRSKVN GQSGRMEFFWTILKPNDAINFESNGNFIAPEYAYKIVKKGDSAIMKSELEYGNCNTKCQ TPMGAINSSMPFHNIHPLTIGECPKYVKSNRLVLATGLRNSPQRESRRKKRGLFGAIAG FIEGGWQGMVDGWYGYHHSNEQGSGYAADKESTQKAIDGVTNKVNSIIDKMNTQFEAVG REFNNLERRIENLNKKMEDGFLDVWTYNAELLVLMENERTLDFHDSNVKNLYDKVRLQL RDNAKELGNGCFEFYHKCDNECMESIRNGTYNYPQYSEEARLKREEISgvklesigtyq ilsiystvasslalaimmaglslwmcsngslqcrici (SEQ ID NO:10). The uppercase bolded amino acids in SEQ ID NO:10 represents the signal peptide that is cleaved during expression. Uppercase and un-bolded amino acids in SEQ ID NO:10 represent the ectodomain. The lowercase and bolded amino acids in SEQ ID NO:10 represent the membrane portion of the HA protein.

In one form, the first and/or second antigen contains a functional fragment of the H5N1 HA protein. For example, the first and/or second antigen can include the H5 hemagglutinin head domain or chimeric HA proteins incorporating H5-specific epitopes. An exemplary amino acid sequence for the H5N1 head domain is represented by SEQ ID NO:12, and is provided below.
Segment of native HA sequence used in construct: L49-K326
Flu A/chicken/Ghana/AVL-763_21VIR7050-39/2021 (H5N1); H5 Clade: 2.3.4.4b)
GISAID Accession Number: EPI2415855
LEKTHNGKLCDLNGVKPLILKDCSVAGWLLGNPMCDEFIRVPEWSYIVERANPANDLCY PGSLNDYEELKHLLSRINHFEKILIIPKSSWPNHETSLGVSAACPYQGAPSFFRNVVWL IKKNDTYPTIKISYNNTNREDLLILWGIHHSNNAEEQTNLYKNPTTYISVGTSTLNQRL VPKIATRSQVNGQRGRMDFFWTILKPDDAIHFESNGNFIAPEYAYKIVKKGDSTIMKSG VEYGHCNTKCQTPVGAINSSMPFHNIHPLTIGECPKYVKSNKGSHHHHHH (SEQ ID NO:12). The amino acid sequence for an exemplary His6-epitope tag is shown in uppercase and bolded letters.

In some forms, the first and/or second antigens may be derived from the same strain of A/H5N1 or from a different strain of A/H5N1. In some forms, the first and/or second antigens can be isolated, recombinantly produced, chemically synthesized, or produced by in vitro transcription and translation systems. The first and/or second antigens can also be selected or engineered to minimize undesired interactions with cross-reactive antibodies, such as H1N1 antibodies, thereby increasing specificity of the assay methods. For example, a recombinant HA protein is provided as SEQ ID NO:4. Recombinant H5N1 HA Protein (526 amino acids)

(SEQ ID NO: 4)
MEKIVLLLAIVSLVKSDQICIGYHANNSTEQVDTIMEKNVTVTHA
QDILEKTHNGKLCDLNGVKPLILRDCSVAGWLLGNPMCDEFTNVP
EWSYIVEKASPANDLCYPGDENDYEELKHLLSRINHFEKIQIIPK
SSWSNHDASSPVSSACPYHGRSSFFRNVVWLIKKNSTYPTIKRSY
NNTNQEDLLVLWGIHHPNDAAEQTKLYQNPTTYISVGTSTLNQRL
VPEIATRPKVNGQSGRMEFFWTILKPIDAINFESNGNFIAPEYAY
KIVKERDSAIMKSELEYGNCNTKCQTPMGAINSSMPFHNIHPLTI
GECPKYVKSNRLVLATGLRNTPQRERRRKKRGLFGAIAGFIEGGW
QGMVDGWYGYHHSNEQGSGYAADKESTQKAIDGVINKVNSIIDKM
NTQFEAVGREFNNLERRIESLNKKMEDGELDVWTYNAELLVLMEN
ERTLDFHDSNVKNLYDKVRLQLRDNAKELGNGCFEFYHKCDNECM
ESVKNGTYDYPQYSEEARLNREEISGVKLESMGTYQ.

The first and/or second antigens may also include variants or mutant forms of the HA protein. For example, the first and/or second antigens may be a variant having at least 70%, at least 80%, at least 85%, at least 90%, at least 95%, at least 98%, at least 99%, such as from about 70% to about 99%, from about 70% to about 95%, from about 70% to about 90%, from about 70% to about 85%, from about 70% to about 80%, from about 70% to about 75% sequence identity to SEQ ID NO:1, SEQ ID NO:2, SEQ ID NO:3, SEQ ID NO:4, SEQ ID NO:7, SEQ ID NO:8, SEQ ID NO:9, SEQ ID NO:10, or SEQ ID NO:12 with and without the signal peptide sequence and/or with or without the ectodomain sequence, or a functional fragment thereof.

The term “variant” refers to a polypeptide or polynucleotide that differs from a reference polypeptide or polynucleotide but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within +2 is preferred, those within +1 are particularly preferred, and those within +0.5 are even more particularly preferred.

Substitution of like amino acids can also be made on the basis of hydrophilicity, particularly, where the biological functional equivalent polypeptide or peptide thereby created is intended for use in immunological forms. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within +2 is preferred, those within +1 are particularly preferred, and those within +0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, forms of the polypeptides can include variants having about 50%, 60%, 70%, 80%, 90%, and 95% sequence identity to the polypeptide of interest.

As used herein, the term “identity,” as known in the art, is a relationship between two or more polypeptide sequences, as determined by comparing the sequences. In the art, “identity” also means the degree of sequence relatedness between polypeptide as determined by the match between strings of such sequences. “Identity” can also mean the degree of sequence relatedness of a polypeptide compared to the full-length of a reference polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

### 2. Detectable Labels

The first and/or second antigen is conjugated, directly or indirectly, to a detectable label, which allows the first and/or second antigen, and any bridging complex formed therewith, to be detected and quantified. “Detectable label” refers to any moiety that can be selectively detected in a screening assay. In some forms, the first antigen can be conjugated to a fluorescent label, a chemiluminescent label, an enzymatic label, a radioactive label, or a nucleic acid label. In some forms, the detectable label is an electrochemiluminescent label, an affinity label, or a combination of two or more label types. In some forms, the second antigen can be conjugated to a solid support, such as a bead, plate, or other surface, or alternatively, to a second label, which may be the same as or different from the label on the first antigen. In some forms, both antigens can be labeled with distinct detectable labels for dual detection or ratiometric analysis. In other forms, one antigen (e.g., the second antigen) can be a binding moiety for a binding partner on a solid support, while the other antigen (e.g., the first antigen) can be labeled for detection, e.g., via separation-based formats such as ELISA or bead-based assays.

Examples include radiolabels, (e.g., ³H, ¹⁴C, ³⁵, ¹²⁵I, ¹³¹I), affinity tags (e.g., biotin/avidin or streptavidin), binding sites for antibodies, metal binding domains, epitope tags, fluorescent or luminescent moieties (e.g., fluorescein and derivatives, green fluorescent protein (GFP), rhodamine and derivatives, lanthanides), colorimetric probe, and enzymatic moieties (e.g., horseradish peroxidase, β-galactosidase, β-lactamase, luciferase, alkaline phosphatase). Additional examples of fluorescent labels include but are not limited to Alexa Fluor dyes and Cy dyes. Examples of chemiluminescent labels include but are not limited to acridinium esters and luminol. Examples of affinity labels include but are not limited to epitope tags such as FLAG, His-tag, and HA-tag, which can be detected directly or via binding partners such as antibodies or metal-chelate reagents.

Suitable detectable labels include, but are not limited to, enzymes, fluorophores, chromophores, chemiluminescent compounds, bioluminescent compounds, nanoparticles, radioactive isotopes, or affinity tags that can be detected using optical, electrochemical, or other analytical detection methods. In some forms, the first antigen is directly labeled using chemical conjugation methods, such as biotinylation, amine-reactive dye labeling, or covalent attachment to an electrochemiluminescent (ECL) tag. For example, the detectable label can be a fluorescent label, e.g., Alexa dyes, FITC, TRITC and green fluorescent protein (GFP). In one example, Iodine-125 can be used as radioactive label. In another example, an enzymatically active label is selected from comprising horseradish peroxidase, glucose oxidase, beta galactosidase, alkaline phosphatase and luciferase. In some forms, the electrochemiluminescent label includes a ruthenium-based label, such as a SULFO-TAG™. SULFO-TAG™ can be detected by applying electrical stimulation at an electrode surface in the presence of a suitable read buffer, thereby generating a measurable electrochemiluminescent signal. In some forms, the SULFO-TAG™ label is conjugated to the first antigen. In these forms, the second antigen can include a binding moiety such as biotin, thereby allowing capture of the bridging complex on a coated surface (e.g., a streptavidin coated surface), and detection of the ECL signal.

Other suitable detectable labels also include nucleic acids, which are compatible for detection via methods for detecting nucleic acids e.g., intercalating dyes, through hybridization, and amplification methods such as PCR.

In some forms, the first and second antigens are conjugated to labels that provide a detectable signal when they are held in proximity to each other, such as through incorporation in the same bridging complex. Examples of such proximity based techniques include fluorescence energy transfer (FRET), amplified luminescent proximity homogenous assay (AlphaScreen), proximity extension assay (PEA) and proximity ligation assay (PLA).

In alternative forms, the first antigen is indirectly labeled through binding to a secondary reagent, such as a labeled antibody or streptavidin-conjugate, that binds to an epitope tag or biotin moiety present on the first antigen. The selection of the detectable label may depend on the desired sensitivity, detection platform, and compatibility with other assay components, and can be tailored to allow quantitative, semi-quantitative, or qualitative detection of the antibody-antigen complex.

### 3. Binding Moieties and Binding Partners

The first and/or second antigen of the bridging complex is linked, either directly or indirectly, to one or more binding moieties. For example, the binding moieties can be used to indirectly attach a label to the first and/or second antigen, as described above. In some forms, a first binding moiety can be attached to (e.g., linked to or conjugated to) the second antigen and facilitates immobilization of the second antigen, or any bridging complex formed therewith, onto a solid phase support. The first binding moiety can be any molecule capable of specific interaction with a complementary binding partner that is immobilized on a solid phase, such as a microtiter plate, bead, sensor surface, or similar substrate.

In one example, the binding moiety is biotin, which allows the second antigen to bind to a streptavidin-coated surface. In another example, the binding moiety may be an epitope tag, such as a His-tag, FLAG-tag, HA-tag, or AviTag, that binds specifically to a complementary anti-tag antibody immobilized on the solid phase. Exemplary epitope tags that can be used include but are not limited to His tags, which typically include six or more, typically consecutive, histidine residues; FLAG tags, which typically include the sequence DYKDDDDK (SEQ ID NO:11); MYC tag for example ILKKATAYIL (SEQ ID NO:13) or EQKLISEEDL (SEQ ID NO: 14). In some forms, the binding moiety may be linked to the second antigen through direct chemical conjugation, genetic fusion during recombinant expression, or through a flexible linker to minimize steric hindrance and preserve antibody binding sites on the second antigen. In one example, the binding moiety is an oligonucleotide that binds specifically to a complementary oligonucleotide immobilized on the solid phase.

The second antigen and binding moiety combination serves to anchor the bridging complex (once formed) onto the solid phase. This allows, in part, for the removal of unbound sample components e.g., unbound antigens through washing steps and facilitates subsequent detection and quantification of the complex based on the detectable label associated with the first antigen. The strength of the detected signal correlates with the amount of bridging complex immobilized on the solid phase, thereby providing a quantitative or semi-quantitative measure of the amount of target antibody present in the sample that is capable of simultaneously binding the first and second antigens. In some forms, the first and second antigens may be identical, closely related variants, or distinct domains derived from the same target antigen, facilitating the detection of bivalent antibodies that recognize either the same epitope on both antigens or distinct epitopes on the first and second antigens.

### C. Blocking Antigen

The methods can further include contacting the sample with an unlabeled blocking antigen that is distinct from the first and second antigens used to form the bridging complex. The purpose of the blocking antigen is to reduce non-specific or cross-reactive binding by antibodies in the sample that may recognize antigens from related but distinct viruses, thereby increasing the specificity of the assay for detecting antibodies specific to the target antigen, e.g., H5N1 antibodies.

In one example, where the target antigen is H5N1 hemagglutinin (HA), the blocking antigen includes a H1N1 protein, peptide, or variant or functional fragment thereof. Because both H5N1 HA and H1N1 HA belong to Group 1 influenza A viruses, they share structural features and antigenic epitopes that can be recognized by cross-reactive antibodies generated in response to prior H1N1 infections. These cross-reactive antibodies may bind to the H5N1 antigens used in the assay, even if the subject was never exposed to H5N1, leading to false positive results.

In some forms, the blocking antigen can be a recombinant or purified H1N1 hemagglutinin (HA) protein, or a fragment or domain thereof. For example, the blocking antigen can be the HA1 subunit or the head domain.

H1 influenza virus strains circulating in humans underwent a major antigenic shift in 2009 with global spread of the H1N1pdm09 virus, from which current H1N1 strains in humans are derived. The blocking antigen can be a recombinant or purified H1 HA protein, or a fragment or domain thereof, selected from a post-2009 pandemic strain that circulated in humans such as Influenza A/Hawaii/70/2019 (H1N1). Other suitable post-2009 HA proteins, or fragments or domains thereof, include but are not limited to ones selected from the H1N1 component of post-2009 vaccines (as selected by the WHO and/or US CDC based on their vaccine composition recommendations for annual influenza vaccines). The blocking antigen can be a recombinant or purified H1 HA protein, or a fragment or domain thereof, selected from a pre-2009 pandemic strain that circulated in humans such as Influenza A/Brisbane/59/2007 (H1N1). Other suitable pre-2009 HA proteins, or fragments or domains thereof, include but are not limited to ones selected from the H1N1 component of pre-2009 vaccines (as selected by the WHO and/or US CDC based on their vaccine composition recommendations for annual influenza vaccines). In some forms, the blocking reagent is a blocker derived from a pre-2009 HA, a blocker derived from a post-2009 HA, or a mixture of a blocker derived from a pre-2009 HA and a blocker derived from a post-2009 HA.

The amino acid sequences for exemplary H1N1 HA proteins are represented by SEQ ID NO:5, SEQ ID NO:6, SEQ ID NO:15, SEQ ID NO:17, or SEQ ID NO:18 which are provided below.
Influenza A virus (strain A/Duck/Australia/749/1980 H1N1)
(UniProt ID: Q9WCE3⋅HEMA_I80A1; 566 amino acids)

(SEQ ID NO: 5)
MEAKLLVLFCTFAALKADTICIGYHANNSTDTVDTVLEKNVTVTH
SVNLLENSHNGKLCSLNGIAPLQLGKCNVAGWLLGNLECDLLLTA
NSWSYIIETSNSENGTCYPGEFIDYEELREQLSSVSSFEKFEIFP
KASSWPNHETTKGVTAACSYLGASSFYRNLLWMTKKGTSYPKLSK
SYTNNKGKEVLVLWGVHHPPTTSEQQTLYQNVDAYVSVGSSKYNR
RFTPEIAARPKVRGQAGKMNYYWTLLDQGDTITFEATGNLIAPWY
AFALNKGSDSGIITSDAPVHNCDTKCQTPYGALNSSLPFQNVHPI
TIGECPKYVKSTKLRMATGLRNVPSIQSRGLFGAIAGFIEGGWTG
MIDGWYGYHHQNEQGSGYAADQKSTQSAIDGITNKVNSVIEKMNT
QFTAVGKEFNNLERRIENLNKKVDDGFLDVWTYNAELLVLLENER
TLDFHDSNVRNLYEKVKSQLRNNAKEIGNGCFEFYHKCDDECMES
VKNGTYDYPKYSEESKLNREEIDGVKLESMGVYQILAIYSTVASS
LVLLVSLGAVSFWMCSNGSLQCRICI.

Influenza A virus (strain A/Swine/Netherlands/12/1985 H1N1)
(UniProt ID: Q9WCE8⋅HEMA_I85A4, 566 amino acids)

(SEQ ID NO: 6)
MEAKLFVLFCAFTTLEADTICVGYHANNSTDTVDTILEKNVTVTH
SVNLLENSHNGKLCSLNGVAPLQLGKCNVAGWILGNPECDLLLTA
NSWSYIIETSDSENGTCYPGEFIDYEELREQLSSVSSFERFEIFP
KANSWPNHETTKGITAACSYSGTLSFYRNLLWIVKRGNSYPKLSK
SYTNNKGKEVLIIWGVHHPPTTSDQQSLYQNADAYVSVGSSKYNR
RFTPEIAARPKVKGQAGRMNYYWILLDQGDTITFEATGNLIAPWY
AFALNKGSGSGIITSDTPVHNCDTKCQTPHGALNSSLPFQNVHPI
TIGECPKYVKSTKLRMATGLRNVPSIQSRGLFGAIAGFIEGGWTG
MIDGWYGYHHQNEQGSGYAADQKSTQIAIDGISNKVNSVIEKMNT
QFTAVGKEFNDLEKRIENLNKKVDDGFLDVWTYNAELLVLLENER
TLDFHDENVRNLYEKVKSQLRNNAKEIGNGCFEFYHKCDDECMES
VKNGTYNYPKYSEESKLNREEIDGVKLESMEVYQILAIYSTVASS
LVLLVSLGAISFWMCSNGSLQCRICI.

HA [(Influenza A virus (A/Brisbane/59/2007(H1N1)]
UniProt ID: D5F1Q8_9INFA

(SEQ ID NO: 15)
MKVKLLVLLCTFTATYADTICIGYHANNSTDTVDTVLEKNVTVTH
SVNLLENSHNGKLCLLKGIAPLQLGNCSVAGWILGNPECELLISK
ESWSYIVEKPNPENGTCYPGHFADYEELREQLSSVSSFERFEIFP
KESSWPNHTVTGVSASCSHNGESSFYRNLLWLTGKNGLYPNLSKS
YANNKEKEVLVLWGVHHPPNIGNQKALYHTENAYVSVVSSHYSRK
FTPEIAKRPKVRDQEGRINYYWTLLEPGDTIIFEANGNLIAPRYA
FALSRGFGSGIINSNAPMDKCDAKCQTPQGAINSSLPFQNVHPVT
IGECPKYVRSAKLRMVTGLRNIPSIQSRGLFGAIAGFIEGGWTGM
VDGWYGYHHQNEQGSGYAADQKSTQNAINGITNKVNSVIEKMNTQ
FTAVGKEFNKLERRMENLNKKVDDGFIDIWTYNAELLVLLENERT
LDFHDSNVKNLYEKVKSQLKNNAKEIGNGCFEFYHKCNDECMESV
KNGTYDYPKYSEESKLNREKIDGVKLESMGVYQILAIYSTVASSL
VLLVSLGAISFWMCSNGSLQCRICI.

HA [Influenza A/Hawaii/70/2019 (H1N1)]
GISAID Accession Number: EPI1617983
MKAILVVLLYTFTTANADTLCIGYHANNSTDTVDTVLEKNVTVTHSVNLLEDKHNGKLC KLRGVAPLHLGKCNIAGWILGNPECESLSTARSWSYIVETSNSDNGTCYPGDFINYEEL REQLSSVSSFERFEIFPKTSSWPNHDSDKGVTAACPHAGAKSFYKNLIWLVKKGNSYPK LNQTYINDKGKEVLVLWGIHHPPTIAAQESLYQNADAYVFVGTSRYSKKFKPEIATRPK VRDQEGRMNYYWTLVEPGDKITFEATGNLVVPRYAFTMERDAGSGIIISDTPVHDCNTT CQTPEGAINTSLPFQNVHPITIGKCPKYVKSTKLRLATGLRNVPSIQSRGLFGAIAGFI EGGWTGMVDGWYGYHHQNEQGSGYAADLKSTQNAIDKITNKVNSVIEKMNTQFTAVGKE FNHLEKRIENLNKKVDDGFLDIWTYNAELLVLLENERTLDYHDSNVKNLYEKVRNQLKN NAKEIGNGCFEFYHKCDNTCMESVKNGTYDYPKYSEEAKLNREKIDGVklestriyqil aiystvasslvlvvslgaisfwmcsngslqcrici (SEQ ID NO:17). The uppercase bolded residues indicate the sequence for the signal peptide that is cleaved during expression. The uppercase unbolded residues represent the ectodomain. The lowercase bolded residues represent the membrane portion of the HA protein.

An exemplary sequence for the head domain for the blocking antigen is represented by SEQ ID NO:18.
Segment of native HA sequence used in construct: L50-K328
Influenza A/Hawaii/70/2019 (H1N1)
GISAID Accession Number: EPI1617983
LEKTHNGKLCDLNGVKPLILKDCSVAGWLLGNPMCDEFIRVPEWSYIVERANPANDLCY PGSLNDYEELKHLLSRINHFEKILIIPKSSWPNHETSLGVSAACPYQGAPSFFRNVVWL IKKNDTYPTIKISYNNTNREDLLILWGIHHSNNAEEQTNLYKNPTTYISVGTSTLNQRL VPKIATRSQVNGQRGRMDFFWTILKPDDAIHFESNGNFIAPEYAYKIVKKGDSTIMKSG VEYGHCNTKCQTPVGAINSSMPFHNIHPLTIGECPKYVKSNKGSHHHHHH (SEQ ID NO: 18). The bolded uppercase letters indicate residues for a His6 epitope tag.

The blocking antigen can also be a variant or mutant form of the HA protein. For example, the blocking antigen may be a variant having at least 70%, at least 80%, at least 85%, at least 90%, at least 95%, at least 98%, at least 99%, such as from about 70% to about 99%, from about 70% to about 95%, from about 70% to about 90%, from about 70% to about 85%, from about 70% to about 80%, from about 70% to about 75% sequence identity to SEQ ID NO:5, SEQ ID NO:6, SEQ ID NO:15, SEQ ID NO:17, or SEQ ID NO:18, with and without the signal peptide sequence and/or with or without the ectodomain sequence, or a functional fragment thereof.

### III. Methods for Detection of H5N1 Antibodies

Disclosed are methods of detecting an influenza virus in a subject. The methods generally include detecting the presence or absence of an antibody in a biological sample e.g., serum sample, from a subject.

An exemplary method includes the following steps: (a) detecting an antibody to a first antigen (also referred to herein as the first modified H5 HA) by contacting a sample obtained from a subject with a first antigen of the antibody linked to a detectable label and a second antigen (also referred to herein as the second modified H5 HA) of the antibody linked to a binding moiety, and (b) detecting the detectable label. The contacting in step (a) generally occurs under conditions suitable for the antibody to bind to the first and second antigens and form a bridging complex.

By detecting antibodies capable of binding to two antigens e.g., H5N1 antigens, the methods increase detection accuracy and reduce false positives. The methods permit the non-invasive assessment of prior viral exposure in human or animal populations, thereby improving surveillance, diagnosis, and risk assessment. The methods can also guide vaccine development, outbreak monitoring, and public health responses by providing reliable data on population-level immunity and cross-species transmission risks.

### A. Sources of Samples

The biological sample for detection of anti-H5N1 antibodies includes but are not limited to bodily fluid, such as blood, serum, or plasma. The sample can also include other bodily fluids such as milk in veterinary applications, or other secretions where circulating antibodies may be present. In some forms, the biological sample is a liquid biopsy derived from blood or serum, which contains circulating antibodies produced in response to prior infection or vaccination. For example, the sample includes bodily fluids that may contain antibodies, including respiratory samples such as nasal swabs, nasopharyngeal washes, bronchoalveolar lavage fluid, and tracheal aspirates, which may contain antibodies secreted into the respiratory tract following H5N1 infection.

The biological sample can be processed using any suitable method for antibody preservation and isolation, including the use of serum separation tubes, anticoagulant-containing collection tubes, and standard blood processing protocols. In some forms, the sample may also undergo pre-treatment, such as the addition of buffers, detergents, or stabilizing agents, to prevent degradation of the proteins.

The subject from whom the biological sample is obtained can be a human or an animal, including mammals, birds, or other species susceptible to H5N1 infection or exposure. Human subjects include but are not limited to individuals at high risk of zoonotic transmission, such as poultry workers, farmers, veterinarians, healthcare personnel, or individuals living in regions experiencing outbreaks of H5N1 influenza. In veterinary applications, the subject includes domestic poultry (e.g., chickens, ducks, turkeys), wild birds, livestock (e.g., cattle), or companion animals that may be exposed to infected birds or contaminated environments. The sample can be collected from symptomatic or asymptomatic subjects to assess prior exposure, immune response following vaccination, or ongoing infection.

### B. Sample Collection and Processing

Blood samples can be collected using standard venipuncture techniques, where blood is drawn into sterile collection tubes containing anticoagulants (such as EDTA, citrate, or heparin) or clot activators for serum preparation. Serum can be isolated by centrifugation, and the resulting serum or plasma can be processed immediately or stored under refrigerated or frozen conditions.

Respiratory samples include, but are not limited to, nasopharyngeal swabs, nasal swabs, oropharyngeal swabs, nasopharyngeal washes, tracheal aspirates, or bronchoalveolar lavage (BAL) fluid. In some forms, nasopharyngeal or nasal swabs are preferred for upper respiratory sampling, while bronchoalveolar lavage (BAL) fluid or tracheal aspirates may be preferred for lower respiratory tract sampling, particularly in individuals requiring intubation or in veterinary applications involving species with respiratory infections.

Swab-based samples can be collected using sterile synthetic fiber swabs, such as flocked swabs, with flexible plastic shafts for nasopharyngeal collection. The swab is inserted into the nostril and gently advanced along the floor of the nasal passage to the posterior nasopharynx, rotated to collect mucus and cellular material, and then placed into a collection tube containing transport medium. In some forms, the transport medium is a viral transport medium (VTM), universal transport medium (UTM), or another clinically approved buffer that preserves antibody integrity during transport and storage. The tube can then securely capped, labeled, and stored under refrigerated conditions (approximately 2° C.-8° C.) until further processing.

For nasopharyngeal washes, a small volume of sterile saline or phosphate-buffered saline (PBS) is instilled into the nostril, and the resulting fluid containing nasal secretions and cellular material is collected into a sterile container. The collected wash fluid is transferred to a transport container, optionally supplemented with protease inhibitors or other stabilizing agents to preserve antibody integrity, and stored at refrigerated temperatures until analysis.

For tracheal aspirates, a suction catheter is inserted into the trachea, and secretions are aspirated into a sterile collection trap. The collected sample can be diluted in sterile saline or transport medium and immediately processed or stored at refrigerated or frozen temperatures, depending on the expected time to analysis. In veterinary applications, tracheal aspirates can be collected directly from livestock or other animals suspected of H5N1 infection using similar techniques.

For bronchoalveolar lavage (BAL), a bronchoscope is advanced into the lower airways, and sterile saline is instilled into a segment of the lung, followed by gentle aspiration to recover lavage fluid containing cells, mucus, and antibodies present within the airway lining fluid. BAL fluid is transferred to a sterile container, optionally supplemented with stabilizers such as BSA, protease inhibitors, or other antibody-preserving agents, and stored at refrigerated (approximately 4° C.) temperatures until processed.

In veterinary applications, biological samples may include milk from lactating animals or oral fluids collected from swabs or drips, particularly in species known to be susceptible to H5N1 infection e.g., cows, pigs.

Following collection, biological samples can be placed in sterile, pre-labeled containers and stored under conditions that preserve antibody stability, such as refrigeration (approximately 4° C.), depending on the anticipated duration before analysis. In some forms, samples can be collected at multiple time points to monitor changes in antibody levels over time.

The sample volume can vary based on the sample type, collection method and the specific requirements of the assay. Exemplary sample volumes range from about 1 mL to about 50 mL, with smaller or larger volumes also contemplated as appropriate for the particular sample type and detection method.

### C. Molecular Analysis

Following the collection of a biological sample, e.g., blood, serum, or a respiratory sample, the sample can be subjected to one or more forms of immunological analysis to detect the presence or absence of antibodies specific to H5N1 influenza virus. These analyses detect and quantify antibodies capable of binding to H5N1 antigens, such as H5N1 HA, to determine whether the subject has been exposed to H5N1 infection or vaccination. The methods utilize a bridging immunoassay, in which the sample is contacted with two H5N1 antigens, one linked to a detectable label and the other linked to a binding partner, under conditions suitable for antibody binding. A bridging complex forms only if antibodies specific to H5N1 are present in the sample, facilitating detection through the detectable label.

The methods can further include a blocking step, in which the sample is contacted with an unlabeled H1N1 antigen to neutralize cross-reactive antibodies that could otherwise bind to H5N1 antigens and generate false positive signals.

The disclosed methods can be performed using standard immunoassay platforms, including but not limited to electrochemiluminescence (ECL) assays, enzyme-linked immunosorbent assays (ELISA), bead-based immunoassays, or similar antibody detection methods. Detection can involve measurement of the signal generated by the detectable label, with the intensity of the signal correlating with the amount of anti-H5N1 antibody present in the sample.

The methods can further include one or more washing steps to remove unbound or non-specifically bound components from the sample, thereby increasing the specificity and signal-to-noise ratio of the assay. Washing steps can be performed after antigen-antibody binding, after formation of the bridging complex, and/or after immobilization of the complex onto a solid phase support. For example, in a plate-based electrochemiluminescence (ECL) immunoassay, the wells may be washed with a buffered detergent solution, such as phosphate-buffered saline with Tween-20 (PBST), to remove unbound antigens, antibodies, and blocking agents. In bead-based immunoassays, washing may include centrifugation or magnetic separation of beads, followed by resuspension in fresh buffer to clear unbound material. In some forms, automated washing stations integrated into immunoassay analyzers may perform reproducible washing cycles across high-throughput sample batches.

In some forms, the ECL reaction can be carried out on a solid substrate e.g., MULTI-SPOT® detection plate (e.g., an MSD ECL platform) in which binding agents (e.g., antibodies) are labeled with a detectable label (e.g., a chemiluminescent tag), such as a SULFO-TAG™. Upon electrical stimulation of the electrode surface, only labeled antibodies in close proximity to the electrode participate in the light-emitting reaction, thereby minimizing background signal and increasing signal-to-noise ratio. Additional details of ECL immunoassays can be found at least in U.S. Pat. Nos. 9,618,510, 10,408,823; 10,241,112; 11,525,825; and 12,411,131, the contents of which are specifically incorporated by reference herein in their entireties.

The bridging immunoassay can detect the presence of anti-H5N1 antibodies in a biological sample with a sensitivity of greater than 60%, such as greater than 70%, greater than 80%, greater than 85%, greater than 90%, greater than 95%, or even greater than 98%. For example, the bridging immunoassay can detect the presence of anti-H5N1 antibodies in a biological sample with a sensitivity between about 60% and about 100%, between about 60% and about 95%, between about 60% and about 90%, between about 60% and about 85%, between about 60% and about 80%, or between about 60% and about 70%. “Sensitivity” refers to the ability of a test to correctly identify true positives, i.e., presence of H5N1 or subjects infected with H5N1. For example, sensitivity can be expressed as a percentage, the proportion of actual positives which are correctly identified as such (e.g., the percentage of test subjects having correctly identified by the test as having H5N1). A test with high sensitivity has a low rate of false negatives, i.e., the cases of H5N1 not identified as such. For example, the sensitivity of detecting anti-H5N1 antibodies can be expressed as the ratio: Sensitivity=True positives/(True positives+false negatives). This equation represents the proportion of actual positive samples correctly identified by the assay.

The bridging immunoassay can detect the presence of anti-H5N1 antibodies in a biological sample with a specificity of greater than 60%, such as greater than 70%, greater than 80%, greater than 85%, greater than 90%, greater than 95%, or even greater than 98%. For example, the bridging immunoassay can detect the presence of anti-H5N1 antibodies in a biological sample with a specificity between about 60% and about 100%, between about 60% and about 95%, between about 60% and about 90%, between about 60% and about 85%, between about 60% and about 80%, or between about 60% and about 70%. “Specificity” refers to the ability of the assay to correctly identify true negatives, i.e., the individuals that have no H5N1. For example, specificity can be expressed as a percentage, the proportion of actual negatives which are correctly identified as such (e.g., the percentage of test subjects not having H5N1 correctly identified by the test as not having H5N1). A test with high specificity has a low rate of false positives, i.e., the cases of individuals not having H5N1 but suggested by the test as having H5N1. In some forms, acceptable specificity of the bridging immunoassay may vary depending on the intended application and the prevalence of H5N1 infection within the target population. For example, in populations for which H5N1 infection is rare, a high specificity is preferred to minimize false positives. In such cases, the immunoassay may achieve a false positive rate of less than about 10%, about 5%, about 2%, about 1%, or even about 0.5%, corresponding to specificities greater than about 90%, about 95%, about 98%, about 99%, or about 99.5%, respectively. In another example, in populations where H5N1 infection is more common, a lower specificity can be acceptable, such as between about 80% and about 90%, between about 70% and about 80%, or between about 60% and about 70%. In some forms, the specificity of the assay can be expressed by the following equation: Specificity=True negatives/(True negatives+False positives).

The presence, absence, or levels of detectable anti-H5N1 antibodies can be used to assess prior exposure to H5N1 virus, detect seroconversion following vaccination, or support epidemiological surveillance efforts. The methods can also be used to monitor antibody levels over time, such as before and after suspected exposure or vaccination, to evaluate immune responses and durability of immunity.

The methods can further include the use of control reagents and control samples to monitor assay performance and provide normalization where appropriate. Controls can include positive controls, negative controls, process controls, and normalization controls.

### D. Detecting the Detectable Label

The methods further include the step of detecting a signal generated by the detectable label attached to the first antigen, wherein the label provides a detectable readout indicative of the formation of a bridging complex between an anti-H5N1 antibody present in the sample, the labeled first antigen, and a second antigen immobilized on a solid phase support. The detection step can include applying appropriate detection conditions to activate the label and measuring the resulting signal using a detection instrument capable of quantifying the signal associated with the captured bridging complex.

The methods for measuring antibodies that bind to influenza hemagglutinin (HA) of the H5 subtype in a sample generally include the following steps: a. forming a bridging complex and b. measuring the formed bridging complex. Generally, the bridging complex includes i. an antibody from the sample having a first and a second binding site for H5 HA; ii. a first modified H5 HA antigen bound to the first binding site; and iii. a second modified H5 HA antigen bound to the second binding site.

An exemplary method includes forming and measuring a bridging complex. The bridging complex can be formed when an H5N1 antibody in the sample, having two binding sites for H5 HA, binds to two modified H5 HA antigens, one at each binding site. For example, the first modified H5 HA antigen binds to the first binding site of the antibody, while the second modified H5 HA antigen binds to the second binding site. The bridging complex is formed when a binding moiety linked to the second modified H5 HA antigen binds with a binding partner, thereby immobilizing the bridging complex on a solid phase support. To measure the formation of the bridging complex, the method includes detecting a signal generated from a detectable label linked to the first modified H5 HA antigen. The presence and quantity of the bridging complex is assessed by measuring the detectable label linked to the first modified H5 HA antigen. The detectable label can be fluorescent, chemiluminescent, enzymatic, or radioactive, depending on the chosen detection platform. The measured signal intensity reflects the extent of bridging complex formation, providing an indication of the presence and activity of H5 HA-specific antibodies in the sample.

In some forms, the amount of the H1N1 HA construct is sufficient to reduce the formation of bridging complexes containing antibodies that are cross-reactive with H1 and H5. For example, the H1N1 HA construct i.e., the H1 HA antigens can competitively bind to one or both of the binding sites on H1N1 HA cross-reactive antibodies, thereby preventing those antibodies from binding to the first and second modified H5 HA antigens and forming undesired bridging complexes. By reducing or blocking the ability of cross-reactive antibodies to bridge between H5N1 HA antigens, the presence of the H1N1 HA construct helps to increase specificity of the assay. In so doing, the measured bridging complexes more accurately reflects the presence and activity of H5N1 HA-specific antibodies rather than antibodies capable of binding both H1N1 and H5N1. The amount of the H1N1 HA construct used is selected to provide effective competition for antibody binding without interfering with the intended detection of H5N1 HA-specific antibodies in the sample.

For example, the detection process may include exposing the solid phase to excitation light, applying an electrical current, introducing a detection reagent or substrate, or applying other activating conditions, depending on the type of label used in the assay. Once activated, the resulting signal, whether luminescent, fluorescent, colorimetric, or electrochemical, is detected and quantified using a compatible detection instrument. In one example, when the label is an ECL tag, the detection method can include applying a voltage to the solid phase support (e.g., a carbon-coated microplate well), triggering an electrochemical reaction that produces light emission. The emitted light can then measured using an electrochemiluminescence reader, such as a Meso Scale Discovery SECTOR™ instrument, with the intensity of the signal correlating with the amount of antibody bound in the sample.

In another example, where the label is a fluorescent dye, the detection method can include illuminating the solid phase with a specific wavelength of light using a fluorescence reader or microplate reader, and measuring the fluorescent emission at a defined wavelength, which reflects the amount of antibody present. In some forms, the detection method may include introducing a colorimetric substrate after the formation of the bridging complex, such as TMB (3,3′,5,5′-Tetramethylbenzidine), followed by measuring the absorbance at a specific wavelength using a spectrophotometer or microplate reader. The color intensity corresponds to the quantity of bound antibody.

In forms where a biotin-labeled antigen can be used, detection may involve adding enzyme-conjugated streptavidin, followed by addition of a chemiluminescent or colorimetric substrate, and subsequent optical detection of the signal.

The detection process may also include automated data acquisition, background correction, normalization to internal controls, and calculation of antibody concentration based on comparison to a standard curve or predefined cut-off value. For example, the level of anti-H5N1 antibodies in the sample can be quantified by measuring the signal generated by a detectable label associated with the formed bridging complex and comparing the measured signal to a standard curve or calibration reference generated using samples containing known concentrations of anti-H5N1 antibodies. The detected signal intensity is generally correlated to the concentration of the antibody in the sample, allowing for quantitative determination of antibody levels, which may be expressed as concentration units, signal intensity, or other suitable metrics.

Detection can be performed using manual benchtop equipment, semi-automated immunoassay platforms, or fully automated high-throughput screening systems, depending on the intended application, throughput requirements, and desired sensitivity. The assay can be adapted various platforms, including but not limited to microtiter plates, bead-based systems, lateral flow devices, and microfluidic cartridges.

### IV. Methods of Diagnosing and Treating Influenza Infections

### A. Diagnostics

In some forms, the disclosed methods are utilized to detect and quantify anti-H5N1 antibodies in a biological sample from a subject, thereby assessing prior exposure, infection status, immune response following vaccination, or overall serological profile related to H5N1 influenza virus.

In some forms, the bridging immunoassay can be performed in a two-step process to further assess the contribution of cross-reactive antibodies to the overall signal. For example, in the first step, a biological sample can be assayed in the absence of a blocking antigen to obtain a total antibody signal. If the sample is positive for H5N1, a second step can be performed, wherein the same sample can be tested in the presence of a blocking antigen, such as an H1N1-derived antigen, to selectively bind and inhibit cross-reactive antibodies.

Detection results obtained using the disclosed immunoassay methods can be compared to reference data to aid in interpreting the results, establishing thresholds, and classifying the subject's antibody response. The reference data can include previously collected antibody levels from individuals with known H5N1 exposure or vaccination history, including both confirmed positive samples and confirmed negative samples. Reference datasets can also include serological profiles from broader epidemiological surveillance programs or historical databases cataloging antibody responses to various influenza strains. Such reference datasets can be curated from clinical studies, surveillance programs, public health repositories, or institutional databases and may be updated over time as new data becomes available. Reference data may also include cut-off values, signal-to-noise thresholds, and antibody titer ranges associated with specific levels of exposure risk.

The analysis of the subject's sample can include qualitative, semi-quantitative, or quantitative comparisons to the reference data. In some forms, the presence or absence of anti-H5N1 antibodies is determined by comparing the detected signal to a predefined cut-off value derived from the reference dataset. In other forms, the detected antibody level is compared to a calibration curve generated from known concentrations of anti-H5N1 antibodies, thereby quantifying the level of the antibody concentration in the sample. This comparative analysis helps distinguish true positive results from low-level, non-specific signals, particularly in samples where cross-reactive antibodies to related influenza subtypes (such as H1N1) may be present.

The methods can include determining whether a subject has been exposed to the first antigen by detecting the presence of a detectable label linked to the first antigen, wherein detection of the label indicates that the first antigen has bound to an antibody in the sample. Formation of a bridging complex between the first antigen, the antibody, and a second antigen confirms that the subject has antibodies capable of binding the first antigen, thereby indicating prior exposure to the first antigen through infection, vaccination, or other antigen contact.

In some forms, the methods include detection of a measurable signal associated with a detectable label linked to the first antigen, wherein the detected signal level exceeds a predetermined threshold level. The threshold level may be established empirically, for example, by analyzing samples from subjects with known exposure to the first antigen (e.g., confirmed H5N1 infection or vaccination) and subjects with no known exposure. The threshold can represent the minimum detectable signal intensity that reliably differentiates positive samples containing antibodies specific to the first antigen from negative samples lacking such antibodies. In some forms, the threshold level accounts for background signal, assay noise, and cross-reactive binding from unrelated antibodies. Detection of a signal at or above the threshold level indicates that sufficient antibody binding has occurred to form a detectable bridging complex, which is indicative of prior exposure to the first antigen. The threshold level can vary based on sample type, assay format, or detection platform and can be periodically reassessed or adjusted based on updated population data or evolving epidemiological trends. In some forms, the threshold level is set higher than the level detected in one or more control samples processed in the same manner as the subject's sample. The control samples may include samples from subjects known to have no prior exposure to the first antigen, subjects from low-risk populations, or archived historical samples collected prior to the introduction or spread of the first antigen in the population. The average signal level detected in the control samples can be used to establish a baseline signal level reflecting background noise, non-specific binding, or low-level cross-reactivity inherent to the assay. The threshold level is then defined as a value exceeding the average control signal by a statistically significant margin, such as a pre-specified number of standard deviations above the mean control level, or by applying a fixed signal-to-noise ratio cutoff.

Following analysis, the results can be summarized into a diagnostic, surveillance, or research report. Such reports may include the detected antibody level, the applied cut-off value, interpretation of the result (e.g., positive, negative, indeterminate), and additional contextual information such as sample type, collection date, and subject demographics if available. The reports can also include graphs, charts, or other visualizations showing the subject's antibody levels in relation to population-level data or historical trends.

The results can be delivered to appropriate recipients, such as clinicians, laboratory personnel, epidemiologists, veterinarians (in the case of animal surveillance), public health agencies, or the subject directly, depending on the context of use. Results may be shared through printed reports, secure electronic records, dedicated laboratory information management systems (LIMS), or other digital platforms. In some forms, automated alerts can be generated for public health authorities if antibody levels consistent with recent or active H5N1 exposure are detected to aid timely outbreak detection and response efforts.

### B. Methods of Treating the Subject

Methods of treating H5N1 infections are provided and can be used alone or in combination with any of the detection methods described herein. For example, the treatment methods may be initiated after a subject is identified as having anti-H5N1 antibodies, indicative of prior or ongoing H5N1 infection, based on the results of the disclosed bridging immunoassay. In some forms, the treatment methods are directed to subjects first selected for treatment based on the results of one or more diagnostic methods. For example, a subject may be identified as having detectable anti-H5N1 antibodies or antibody levels exceeding a defined threshold, indicating likely exposure to H5N1 virus, and subsequently initiated on antiviral therapy, immunomodulatory therapy, supportive care, or a combination thereof.

In some forms, the methods include reviewing the diagnostic results from H5N1 antibody testing and providing an appropriate treatment protocol tailored to the subject's infection status, risk factors, and clinical condition. Additionally, or alternatively, the methods may include avoiding unnecessary therapies or treatments in subjects where the absence of anti-H5N1 antibodies suggests no evidence of exposure or infection. For example, a subject with a confirmed positive H5N1 antibody result may be treated with antiviral agents targeting influenza A viruses, such as HA inhibitors (e.g., arbidol) and/or neuraminidase inhibitors (e.g., oseltamivir or zanamivir), while a subject without detectable H5N1 antibodies may instead be monitored for symptoms or offered preventive measures, such as vaccination or prophylactic antivirals.

In some forms, treatment may also be adjusted based on the subject's antibody levels over time, such as rising or sustained antibody titers, which may indicate ongoing or recent infection, or declining antibody titers, which may indicate resolving infection or waning immunity. The treatment regimen can be repeated or modified as necessary based on clinical progression, antibody trends, or emerging symptoms. Treatment may be administered once daily, twice daily, or at other intervals, depending on the severity of infection, the therapeutic agent used, and clinical guidelines.

The therapeutic agents may be administered via any suitable route, including but not limited to oral, intravenous, intramuscular, intranasal, or subcutaneous administration, depending on the specific therapy and the subject's clinical status. Systemic treatments, such as oral antivirals or immunomodulators, may be used to control viral replication and modulate the host immune response, while inhaled or intranasal therapies may be used to deliver antivirals or other agents directly to the respiratory tract, particularly in cases where primary infection is localized to the upper or lower respiratory system.

One or more therapeutic compositions can be administered via multiple routes at the same or different times, depending on the severity of infection, the presence of co-infections, or the subject's underlying health conditions. Treatment effectiveness can be measured using clinical and laboratory indicators, including resolution of symptoms, reduction in viral load (if tested), and stabilization or reduction in inflammatory markers. In some forms, changes in antibody levels, such as a decline in H5N1-specific antibodies after treatment, may also be used as an indirect indicator of reduced viral antigen exposure or successful viral clearance.

The improvement in clinical condition or biomarker levels (if applicable) may be quantified, such as by a reduction in viral load, reduction in inflammatory cytokines, or improvement in respiratory function. Improvement may also be assessed subjectively based on symptom relief or objectively based on clinical scoring systems, imaging studies (e.g., chest X-ray improvement), or other validated assessment tools.

### V. Kits

Kits for performing the bridging immunoassay are also provided. The kits can include reagents and materials for incorporating the immunoassay into an existing assay such as an ELISA. Such kits generally include reagents for performing the assay. For example, the kits can include peptides/proteins for the first and second antigens (e.g., H5N1 antigens), and/or peptides/proteins for the blocking antigens (e.g., H1N1 antigens). Optionally, the kits can further include sample diluents, buffers (e.g., wash buffers, blocking buffer), substrate solutions, and detection reagents. The kit can also include plates, beads, or other solid-phase supports, optionally pre-coated with a binding partner for immobilizing the second antigen. Optionally, the kits can also include positive and negative control samples for assay validation. Optionally, the kits can also include instructions for assay setup, incubation conditions, and data interpretation.

In some forms, the kits can be formulated as lateral flow assays, such as rapid diagnostic tests for point-of-care applications. A “lateral flow” assay is a device intended to detect the presence (or absence) of a target analyte in sample in which the test sample flows along a solid substrate via capillary action. The term “membrane” as used herein refers to a solid substrate with sufficient porosity to allow movement of antibodies or aptamers bound to analyte by capillary action along its surface and through its interior. The term “membrane strip” or “test strip” refers to a length and width of membrane sufficient to allow separation and detection of analyte. The term “application point” is the position on the membrane where a fluid can be applied. The term “capture particle” refers to a particle coated with a plurality of capture agents. In preferred forms, the capture particle is immobilized in a defined capture zone. The term “capture zone” refers to a point on a membrane strip at which one or more capture agents are immobilized.

In an exemplary lateral flow assay, the test sample flows along a solid substrate via capillary action. An exemplary lateral flow device includes a solid substrate, such as a membrane strip, having an application point, an optional conjugate zone, a capture zone, and an absorbent zone (e.g., a wicking pad). Binding agents are optionally present in the conjugate zone. Capture agents are immobilized in the capture zone, which preferably contains a plurality of capture lines for detecting captured analyte (capture complex). The sample pad facilitates the controlled flow of the test solution, which migrates to the conjugate pad where nanoparticles labelled with antibodies (or any binding partner for the analyte in the sample) are stored. For example, the binding agent in the capture zone can be an antibody or biomarker binding fragment thereof, or an aptamer. If the target analyte is present, the labelled antibodies will bind to it and continue to migrate to the detection pad, whereupon the materials are captured by immobilized antibodies at a test line (T-line) to form a coloured strip while a subsequent control line (C-line) is used to colorimetric ally indicate that the solution has sufficiently migrated. Finally, the absorbent pad absorbs excess sample.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

The disclosed methods, compositions and kits can be further understood through one or more of the following non-limiting paragraphs.

Paragraph 1. A method for measuring antibodies that bind to influenza hemagglutinin (HA) of the H5 subtype in a sample; the method including:
a. forming a bridging complex containing:
   i. an antibody from the sample having a first and a second binding site for H5 HA;
   ii. a first modified H5 HA antigen bound to the first binding site; and
   iii. a second modified H5 HA antigen bound to the second binding site; and
b. measuring the formed bridging complex.

Paragraph 2. The method of paragraph 1, wherein step (a) includes contacting the antibody with the first and/or second modified H5 HA antigens in a solution that contains a soluble H1 HA construct.

Paragraph 3. The method of paragraph 2, wherein the amount of the H1 HA construct is sufficient to reduce the formation of bridging complexes comprising antibodies that are cross-reactive with H1 and H5.

Paragraph 4. The method of paragraph 2 or paragraph 3, wherein the H1 HA construct includes the full ectodomain of an H1 HA.

Paragraph 5. The method of any one of paragraphs 2 to 4, wherein the H1 HA construct includes the head domain of an H1 HA.

Paragraph 6. The method of any one of paragraphs 1 to 5, wherein the first and second modified H5 HAs include a first and a second detectable label, respectively, and measuring the formed bridging complex includes generating a signal that is dependent on the proximity of the first and second detectable labels.

Paragraph 7. The method of any one of paragraphs 1 to 5, wherein the first modified H5 HA antigen includes a first detectable label and the second modified H5 HA antigen is immobilized on a solid phase support, and
wherein step (b) includes measuring the first detectable label on the solid support.

Paragraph 8. The method of any one of paragraphs 1 to 5, wherein:
the first modified H5 HA antigen includes a first detectable label,
the second modified H5 HA antigen includes a binding moiety, and the step of measuring the formed bridging complex includes binding the binding moiety to a binding partner of the binding moiety on a solid phase support, and measuring the first detectable label on said solid support.

Paragraph 9. The method of any one of paragraphs 1-8, wherein the step of forming the bridging complex is carried out in solution, and the bridging complex is subsequently immobilized on the solid support by the binding of the binding moiety to the binding partner.

Paragraph 10. The method of any one of paragraphs 7 to 9, wherein the detectable label on the solid support undergoes a wash step to remove free detectable label in solution, prior to measuring the detectable label.

Paragraph 11. The method of any one of paragraphs 1 to 10, wherein the first and second modified H5 HAs antigens do not include HA domains involved in HA trimerization.

Paragraph 12. The method of any one of paragraphs 1 to 11, wherein the HA amino acid sequences included in the modified H5 HAs are amino acids from a head domain.

Paragraph 13. The method of any one of paragraphs 1 to 10, wherein the HA amino acid sequences included in the modified H5 HAs are amino acids from the HA1 HA fragment.

Paragraph 14. The method of any one of paragraphs 1 to 13, further including analyzing one or more calibration standards with pre-determined H5 antibody activity values, generating a calibration equation based on the measurements from the calibration standards, and applying the calibration equation to convert the detected level of the bridging complex in the sample into an H5 antibody activity value.

Paragraph 15. The method of any one of paragraphs 1 to 14, wherein the sample is selected from the group of blood, plasma, serum, milk, and a combination thereof.

Paragraph 16. The method of any one of paragraphs 1 to 15, wherein the sample is obtained from a mammal or a bird.

Paragraph 17. The method of paragraph 16, wherein the mammal is a human or a cow.

Paragraph 18. The method of paragraph 16, wherein the sample is obtained from a non-human animal.

Paragraph 19. A method of detecting an antibody to a first antigen including
contacting a sample with a first antigen of the antibody linked a detectable label and second antigen of the antibody linked to a binding moiety under conditions suitable for the antibody to bind to the first and second antigens and form a bridging complex included thereof,
optionally contacting the sample with an unlabeled blocking antigen that is not the same as the first and second antigens; and
detecting the detectable label.

Paragraph 20. The method of paragraph 19, including contacting the second antigen with a binding partner that binds the binding moiety.

Paragraph 21. The method of paragraph 20, wherein the binding partner is immobilized on a surface.

Paragraph 22. The method of any one of paragraphs 19-21, wherein the first and second antigens are selected from peptides, proteins, carbohydrates, lipids, and combinations thereof.

Paragraph 23. The method of any one of paragraphs 19-22, wherein the first and second antigens are the same.

Paragraph 24. The method of any one of paragraphs 19-23, wherein the blocking antigen is selected to reduce or prevent binding of cross-reactive antibodies that recognize epitopes shared between the first and second antigens and the blocking antigen; optionally wherein the blocking antigen reduces the formation of bridging complexes formed by cross-reactive antibodies.

Paragraph 25. The method of any one of paragraphs 19-24, wherein the detectable label is selected from the group of fluorescent dyes, enzymes, electrochemiluminescent labels, chemiluminescent labels, biotin, affinity tags, and a combination thereof.

Paragraph 26. The method of any one of paragraphs 19-25, wherein the detectable label is selected from the group of fluorescein, Alexa Fluor dyes, Cy dyes, horseradish peroxidase (RP), alkaline phosphatase (AP), acridinium esters, luminol, FLAG, His-tag, HA-tag, and a combination thereof.

Paragraph 27. The method of any one of paragraphs 19-26, wherein the binding partners are selected from the group of biotin and avidin, biotin and streptavidin, biotin and neutravidin, hapten and anti-hapten antibody pairs, digoxigenin and anti-digoxigenin antibodies, fluorescein and anti-fluorescein antibodies, and complementary oligonucleotides.

Paragraph 28. The method of any one of paragraphs 19-27, wherein detection includes measuring the level of the detectable label.

Paragraph 29. The method of any one of paragraphs 19-28, further including one or more washing steps.

Paragraph 30. The method of any one of paragraphs 19-29, wherein some antibodies in the sample are cross-reactive to the first, second, and blocking antigens.

Paragraph 31. The method of any one of paragraphs 19-30, wherein the first and second antigens are an H5N1 HA protein or a fragment thereof and/or the blocking antigen is an H1N1 HA protein or a fragment thereof.

Paragraph 32. The method of any one of paragraphs 19-31, wherein the sample is from a subject.

Paragraph 33. The method of paragraph 32, wherein the subject is selected from the group of a human, a domestic animal, a livestock animal, a companion animal, a wild bird, a poultry animal, and combinations thereof.

Paragraph 34. A method of determining if a subject has been exposed to a first antigen including:
detecting an antibody the binds to the first antigen in a sample from the subject according to the method of any one of paragraphs 19-33, and
determining that the subject has been exposed to the first antigen when the detectable label is detected.

Paragraph 35. The method of paragraph 34, wherein determining that the subject has been exposed to the first antigen when the detectable label is detected at a level that exceeds a threshold level.

Paragraph 36. The method of paragraph 35, wherein the threshold level if higher than the level detected in a control sample or average of control samples analyzed in the same manner as the sample from the subject.

Paragraph 37. The method of paragraphs 35 and 36, wherein the threshold level is a predetermined standard.

Paragraph 38. A method of diagnosing a subject with a disease or disorder associated with exposure the first antigen including determining the subject has been exposed to the first antigen according to the method of any one of paragraphs 34-37.

Paragraph 39. The method of paragraph 38, further including treating the subject for the disease or disorder.

Paragraph 40. A method of determining if a subject has been vaccinated against the first antigen including determining the subject has been exposed to the first antigen according to the method of any one of paragraphs 34-39.

Paragraph 41. The method of any one of paragraphs 2-5, wherein the H1 HA construct includes a mixture of two or more H1 HA proteins or fragments thereof.

Paragraph 42. The method of paragraph 6, wherein the first modified H5 HA antigen includes a first detectable label or the second modified H5 HA antigen includes a second detectable label,
optionally wherein the first detectable label and the second detectable label are different; and
wherein measuring the formed bridging complex includes generating a signal that is dependent on the proximity of the first detectable label and the second detectable label.

Paragraph 43. The method of paragraph 42, wherein the first detectable label and/or the second detectable label is selected from electrochemiluminescent labels, fluorescent labels, chemiluminescent labels, enzymatic labels, affinity labels, and combinations thereof.

Paragraph 44. The method of paragraph 42 or paragraph 43, wherein the first detectable label and/or the second detectable label is selected from fluorescein, Alexa Fluor dyes, Cy dyes, horseradish peroxidase (HRP), alkaline phosphatase (AP), acridinium esters, luminol, FLAG, His-tag, HA-tag, SULFO-TAG, and combinations thereof.

Paragraph 45. The method of paragraph 7, wherein the first detectable label is selected from the group of an electrochemiluminescent label, a fluorescent label, a chemiluminescent label, an enzymatic label, an affinity label, and combinations thereof.

Paragraph 46. The method of paragraph 7 or paragraph 45, wherein the first detectable label is selected from the group of fluorescein, Alexa Fluor 488, Alexa Fluor 647, Cy3, Cy5, horseradish peroxidase (HRP), rhodamine, alkaline phosphatase (AP), 0-galactosidase, acridinium esters, Luciferase, luminol, FLAG, His-tag, HA-tag, SULFO-TAG, and combinations thereof.

Paragraph 47. The method of paragraph 8, wherein the binding moiety is selected from biotin, affinity tags, haptens, and combinations thereof.

Paragraph 48. The method of paragraph 47, wherein the binding partner includes streptavidin, avidin, or neutravidin immobilized on the solid phase support.

Paragraph 49. The method of paragraph 8, wherein the binding moiety includes biotin and the binding partner includes streptavidin.

Paragraph 50. The method of paragraph 1, wherein the first and second modified H5 HA antigens include one or more amino acid sequences selected from SEQ ID NO:1, SEQ ID NO:2, SEQ ID NO:3, SEQ ID NO:7, SEQ ID NO:8, SEQ ID NO:9, SEQ ID NO:10, and SEQ ID NO:12 or one or more amino acid sequences having at least 70%, at least 80%, at least 85%, at least 90%, at least 95%, at least 98%, at least 99%, such as from about 70% to about 99%, from about 70% to about 95%, from about 70% to about 90%, from about 70% to about 85%, from about 70% to about 80%, from about 70% to about 75% sequence identity to SEQ ID NO:1, SEQ ID NO:2, SEQ ID NO:3, SEQ ID NO:4, SEQ ID NO:7, SEQ ID NO:8, SEQ ID NO:9, SEQ ID NO:10, or SEQ ID NO:12.

Paragraph 51. The method of paragraph 12, wherein the first and second modified H5 HA antigens include one or more amino acid sequences from the head domain of an H5 HA (H5 HA amino acid sequences).

Paragraph 52. The method of paragraph 51, wherein the H5 HA amino acid sequence is SEQ ID NO:12 or an amino acid sequence having at least 70%, at least 80%, at least 85%, at least 90%, at least 95%, at least 98%, at least 99%, such as from about 70% to about 99%, from about 70% to about 95%, from about 70% to about 90%, from about 70% to about 85%, from about 70% to about 80%, from about 70% to about 75% sequence identity to SEQ ID NO:12.

Paragraph 53. The method of paragraph 13, wherein the H1N1 HA antigen has an amino acid sequence selected from SEQ ID NO:5, SEQ ID NO:6, SEQ ID NO:15, SEQ ID NO:17, and SEQ ID NO:18, or an amino acid sequence having at least 70%, at least 80%, at least 85%, at least 90%, at least 95%, at least 98%, at least 99%, such as from about 70% to about 99%, from about 70% to about 95%, from about 70% to about 90%, from about 70% to about 85%, from about 70% to about 80%, from about 70% to about 75% sequence identity to SEQ ID NO:5, SEQ ID NO:6, SEQ ID NO:15, SEQ ID NO:17, and SEQ ID NO:18.

Paragraph 54. The method of paragraph 53, wherein the H1N1 HA antigen has the amino acid sequence SEQ ID NO:18 or an amino acid sequence having at least 70%, at least 80%, at least 85%, at least 90%, at least 95%, at least 98%, at least 99%, such as from about 70% to about 99%, from about 70% to about 95%, from about 70% to about 90%, from about 70% to about 85%, from about 70% to about 80%, from about 70% to about 75% sequence identity to SEQ ID NO:18.

Paragraph 55. A method of determining if a subject has been exposed to H5N1 including measuring the formed bridging complex in a sample obtained from the subject.

Paragraph 56. The method of paragraph 55, wherein determining that the subject has been exposed to the first antigen when the detectable label is detected at a level that exceeds a threshold level.

Paragraph 57. The method of paragraph 56, wherein the threshold level if higher than the level detected in a control sample or average of control samples analyzed in the same manner as the sample from the subject.

Paragraph 58. The method of paragraph 56 or paragraph 57, wherein the threshold level is a predetermined standard.

Paragraph 59. A method of diagnosing a subject with an infection caused by H5N1 including determining the subject has been exposed to H5N1 according to the method of any one of paragraphs 55-58.

Paragraph 60. A composition, kit, method, or reagent as described herein including, but not limited to, the text, drawings, or combination thereof.

### EXAMPLES

### Example 1: Serological Screening for Human H5N1 Influenza Infections while Blocking H1N1 Antibody Activity

### Materials and Methods

A set of H5-naïve de-identified archived serum samples from 76 human donors was purchased from a commercial vendor (BioIVT). Four archived pooled positive control serum samples, collected from studies of H5 vaccines based on the A/Vietnam/1203/04 and A/Indonesia/5/2005 strains, were provided by BEI Resources. A recombinant construct with the HA head domain (amino acids 33-310) of the clade 2.3.4.4b H5N1 strain A/Ghana/AVL-763_21VIR7050-39/21 was used as the antigen for the H5 antibody assay. Blocking of anti-H1 antibodies was performed using recombinant H1 from the H1N1 strain A/Hawaii/70/19.

Testing for anti-H5 antibodies was done using a bridging serology format that requires antibodies to simultaneously bind two labeled antigens to generate signal (FIG. 1). This creates a high stringency condition that makes it the gold standard for measurement of anti-drug antibody (ADA) responses in biologic drug studies. The assay included electrochemiluminescence (ECL) detection using reagents and consumables from Meso Scale Discovery (MSD). The protocol included (i) forming bridging complexes by incubating 1:20 diluted samples with a solution containing biotin-labeled H5 antigen and H5 antigen labeled with an ECL tag; (ii) capturing the bridging complexes by incubating the mixture in pre-blocked MSD GOLD 96-well Small Spot Streptavidin SECTOR Plates; and (iii) washing the plate, adding MSD GOLD Read Buffer B and measuring ECL from the bound tag in a MESO SECTOR S 600 MM plate reader. The measurements were repeated with and without 20 μg/mL H1 HA in the sample diluent to characterize anti-H1 interference. Raw ECL signals were normalized against a calibration curve generated with a rabbit H5 antiserum and were reported in arbitrary units (AU) per mL.

### Results

Table 1 shows the results from the H5 bridging assay. Results for testing of 76 presumed H5-naïve serum samples (SampleType=Presumed Naïve) and 4 pooled positive serum samples from H5 vaccine studies (SampleType=Vaccinated). For the vaccine study samples, the VaccineInfo column provides the H5N1 vaccine strain (A/Viet. for A/Vietnam/1203/04 and A/Indon. for A/Indonesia/5/2005). The No Blocking and H1 Blocking fields provided the measured anti-H5 antibody activity in each sample in the absence or presence of free H1 HA as a blocking agent for anti-H1 antibody activity. Anti-H5 antibody activity is reported in arbitrary units per mL (AU/mL) relative to a rabbit polyclonal calibration standard. Values below the assay detection limit after adjusting for the 20× sample dilution were assigned the value of the adjusted detection limit (0.2 AU/mL).

TABLE 1
Data for Serum Sample Testing with H5 Bridging Assay

| | | | Anti-H5 | |
|---|---|---|---|---|
| | | | Activity (AU/mL) | |
| | | | No | H1 |
| SampleID | SampleType | VaccineInfo | Blocking | Blocking |
| Neg-Sample-01 | Presumed Naïve | NA | 1.22 | 0.21 |
| Neg-Sample-02 | Presumed Naïve | NA | 0.73 | 0.20 |
| Neg-Sample-03 | Presumed Naïve | NA | 0.20 | 0.20 |
| Neg-Sample-04 | Presumed Naïve | NA | 0.20 | 0.20 |
| Neg-Sample-05 | Presumed Naïve | NA | 0.20 | 0.20 |
| Neg-Sample-06 | Presumed Naïve | NA | 0.20 | 0.20 |
| Neg-Sample-07 | Presumed Naïve | NA | 0.20 | 0.20 |
| Neg-Sample-08 | Presumed Naïve | NA | 0.22 | 0.20 |
| Neg-Sample-09 | Presumed Naïve | NA | 0.20 | 0.20 |
| Neg-Sample-10 | Presumed Naïve | NA | 1.14 | 0.39 |
| Neg-Sample-11 | Presumed Naïve | NA | 0.20 | 0.20 |
| Neg-Sample-12 | Presumed Naïve | NA | 0.49 | 0.32 |
| Neg-Sample-13 | Presumed Naïve | NA | 0.20 | 0.20 |
| Neg-Sample-14 | Presumed Naïve | NA | 0.20 | 0.20 |
| Neg-Sample-15 | Presumed Naïve | NA | 0.49 | 0.20 |
| Neg-Sample-16 | Presumed Naïve | NA | 0.48 | 0.33 |
| Neg-Sample-17 | Presumed Naïve | NA | 10.46 | 1.80 |
| Neg-Sample-18 | Presumed Naïve | NA | 0.30 | 0.20 |
| Neg-Sample-19 | Presumed Naïve | NA | 1.14 | 0.67 |
| Neg-Sample-20 | Presumed Naïve | NA | 0.53 | 0.20 |
| Neg-Sample-21 | Presumed Naïve | NA | 0.34 | 0.20 |
| Neg-Sample-22 | Presumed Naïve | NA | 0.20 | 0.20 |
| Neg-Sample-23 | Presumed Naïve | NA | 0.20 | 0.20 |
| Neg-Sample-24 | Presumed Naïve | NA | 71.88 | 4.40 |
| Neg-Sample-25 | Presumed Naïve | NA | 0.20 | 0.20 |
| Neg-Sample-26 | Presumed Naïve | NA | 1.13 | 0.93 |
| Neg-Sample-27 | Presumed Naïve | NA | 0.20 | 0.20 |
| Neg-Sample-28 | Presumed Naïve | NA | 0.20 | 0.20 |
| Neg-Sample-29 | Presumed Naïve | NA | 0.20 | 0.20 |
| Neg-Sample-30 | Presumed Naïve | NA | 0.20 | 0.20 |
| Neg-Sample-31 | Presumed Naïve | NA | 0.20 | 0.20 |
| Neg-Sample-32 | Presumed Naïve | NA | 0.20 | 0.20 |
| Neg-Sample-33 | Presumed Naïve | NA | 0.35 | 0.20 |
| Neg-Sample-34 | Presumed Naïve | NA | 0.20 | 0.20 |
| Neg-Sample-35 | Presumed Naïve | NA | 1.74 | 0.90 |
| Neg-Sample-36 | Presumed Naïve | NA | 0.20 | 0.20 |
| Neg-Sample-37 | Presumed Naïve | NA | 0.20 | 0.20 |
| Neg-Sample-38 | Presumed Naïve | NA | 0.20 | 0.20 |
| Neg-Sample-39 | Presumed Naïve | NA | 0.20 | 0.20 |
| Neg-Sample-40 | Presumed Naïve | NA | 1.97 | 0.91 |
| Neg-Sample-41 | Presumed Naïve | NA | 0.20 | 0.20 |
| Neg-Sample-42 | Presumed Naïve | NA | 0.20 | 0.20 |
| Neg-Sample-43 | Presumed Naïve | NA | 0.20 | 0.20 |
| Neg-Sample-44 | Presumed Naïve | NA | 0.20 | 0.20 |
| Neg-Sample-45 | Presumed Naïve | NA | 0.20 | 0.20 |
| Neg-Sample-46 | Presumed Naïve | NA | 2.17 | 0.71 |
| Neg-Sample-47 | Presumed Naïve | NA | 0.20 | 0.20 |
| Neg-Sample-48 | Presumed Naïve | NA | 0.20 | 0.20 |
| Neg-Sample-49 | Presumed Naïve | NA | 0.20 | 0.20 |
| Neg-Sample-50 | Presumed Naïve | NA | 0.20 | 0.20 |
| Neg-Sample-51 | Presumed Naïve | NA | 0.20 | 0.20 |
| Neg-Sample-52 | Presumed Naïve | NA | 0.20 | 0.20 |
| Neg-Sample-53 | Presumed Naïve | NA | 0.20 | 0.20 |
| Neg-Sample-54 | Presumed Naïve | NA | 0.20 | 0.20 |
| Neg-Sample-55 | Presumed Naïve | NA | 0.20 | 0.20 |
| Neg-Sample-56 | Presumed Naïve | NA | 0.20 | 0.20 |
| Neg-Sample-57 | Presumed Naïve | NA | 0.20 | 0.20 |
| Neg-Sample-58 | Presumed Naïve | NA | 0.20 | 0.20 |
| Neg-Sample-59 | Presumed Naïve | NA | 3.46 | 3.11 |
| Neg-Sample-60 | Presumed Naïve | NA | 1.24 | 1.35 |
| Neg-Sample-61 | Presumed Naïve | NA | 0.20 | 0.20 |
| Neg-Sample-62 | Presumed Naïve | NA | 0.20 | 0.20 |
| Neg-Sample-63 | Presumed Naïve | NA | 21.63 | 2.57 |
| Neg-Sample-64 | Presumed Naïve | NA | 0.20 | 0.20 |
| Neg-Sample-65 | Presumed Naïve | NA | 0.20 | 0.20 |
| Neg-Sample-66 | Presumed Naïve | NA | 0.20 | 0.20 |
| Neg-Sample-67 | Presumed Naïve | NA | 0.20 | 0.20 |
| Neg-Sample-68 | Presumed Naïve | NA | 0.20 | 0.20 |
| Neg-Sample-69 | Presumed Naïve | NA | 0.20 | 0.20 |
| Neg-Sample-70 | Presumed Naïve | NA | 0.20 | 0.20 |
| Neg-Sample-71 | Presumed Naïve | NA | 0.20 | 0.20 |
| Neg-Sample-72 | Presumed Naïve | NA | 1.61 | 0.29 |
| Neg-Sample-73 | Presumed Naïve | NA | 0.20 | 0.20 |
| Neg-Sample-74 | Presumed Naïve | NA | 0.20 | 0.20 |
| Neg-Sample-75 | Presumed Naïve | NA | 0.20 | 0.20 |
| Neg-Sample-76 | Presumed Naïve | NA | 0.20 | 0.20 |
| Pos-Sample-01 | Vaccinated | A/Indon., | 29.28 | 25.29 |
| | | HAI = 20 | | |
| Pos-Sample-02 | Vaccinated | A/Indon., | 39.59 | 26.32 |
| | | HAI = 80 | | |
| Pos-Sample-03 | Vaccinated | A/Viet., | 55.42 | 35.52 |
| | | HAI = 80 | | |
| Pos-Sample-04 | Vaccinated | A/Viet., | 124.13 | 117.23 |
| | | HAI = 320 | | |

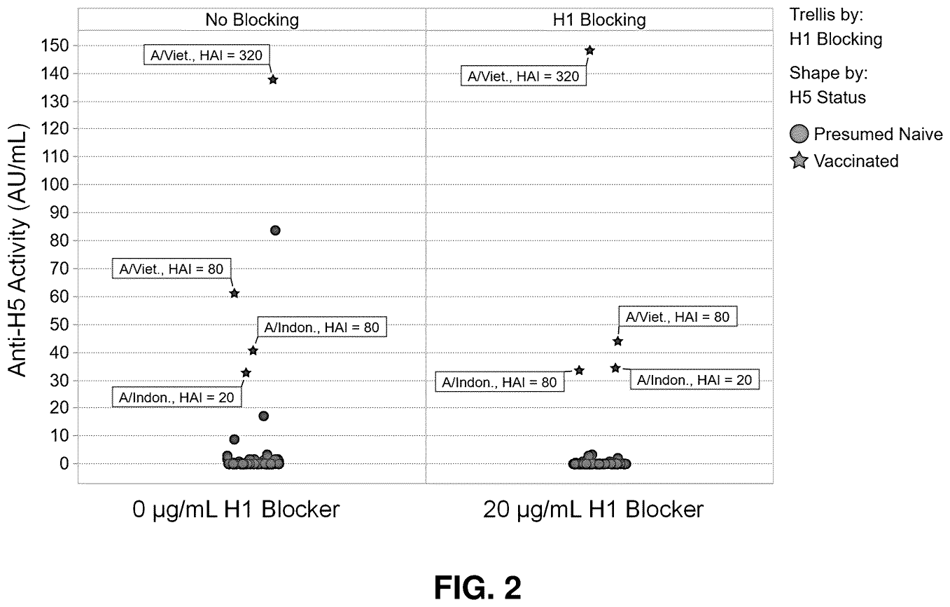

FIG. 2 (left panel) shows the antibody levels as measured using the H5 bridging serology assay without blocking anti-H1 activity. The four positive control samples had concentrations ranging from 29 to 124 AU/mL. Most of the negative samples (73 of 76) were clustered, with concentrations less than 5 AU/mL. However, three outlier negative samples had measured concentrations similar to the positive samples, presumably due to cross-reactive anti-H1 activity. FIG. 2 (right panel) shows that blocking anti-H1 activity by adding soluble H1 decreases the measured concentrations of the negative outliers into the cluster below 5 AU/mL, while maintaining the separation of the positive samples.

### Discussion and Conclusions

The results demonstrate the feasibility of using an H5 bridging serology assay, with H1 blocking, to identify H5 influenza seroconversion in humans. The identification of 3 out of 76 negative samples (3.9%) with high anti-H5 activity in the absence of H1 blocking demonstrates that the assay was effective at addressing anti-H1 cross-reactivity.

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

### REFERENCES

1. Mellis A M, Coyle J, Marshall K E, et al. Serologic Evidence of Recent Infection with Highly Pathogenic Avian Influenza A(H5) Virus Among Dairy Workers—Michigan and Colorado, June-August 2024. MMWR Morb Mortal Wkly Rep. 2024; 73(44):1004-1009.
2. Leonard J, Harker E J, Szablewski C M, et al. Seroprevalence of Highly Pathogenic Avian Influenza A(H5) Virus Infections Among Bovine Veterinary Practitioners—United States, September 2024. MMWR Morb Mortal Wkly Rep. 2025; 74(4):50-52.
3. Corti D, Suguitan A L, Pinna D, et al. Heterosubtypic neutralizing antibodies are produced by individuals immunized with a seasonal influenza vaccine. J Clin Invest. 2010; 120(5):1663-1673.
4. Carreño J M, Strohmeier S, Kirkpatrick Roubidoux E, Hai R, Palese P, Krammer F. H1 Hemagglutinin Priming Provides Long-Lasting Heterosubtypic Immunity against H5N1 Challenge in the Mouse Model. mBio. 2020; 11(6):10.1128/mbio.02090-20.
5. Gioia C, Castilletti C, Tempestilli M, et al. Cross-subtype Immunity against Avian Influenza in Persons Recently Vaccinated for Influenza—Volume 14, Number 1—January 2008—Emerging Infectious Diseases journal—CDC.
6. Pöhler A, Emrich T, Jordan G, et al. Comparison of Assay Formats Used for the Detection of Pre-Existing Anti-Drug Antibodies Against Monoclonal Antibodies. Bioanalysis. 2022; 14(13):923-933.
7. Aebishcher, et al. (2025), Development of a multi-species luciferase-based double antigen ELISA for the detection of antibodies against Influenza A virus H5 clade 2.3.4.4b, bioRxiv, 10.64898/2026.01.05.697617.

Those skilled in the art will recognize, or be able to ascertain, using no more than routine experimentation, many equivalents to the specific embodiments of the method and compositions described herein. Such equivalents are intended to be encompassed by the following claims.

We claim:

1. A method for measuring antibodies that bind to influenza hemagglutinin (HA) of the H5 subtype in a sample; the method comprising:
a. forming a bridging complex comprising:
i. an antibody from the sample having a first and a second binding site for H5 HA;
ii. a first modified H5 HA antigen bound to the first binding site; and
iii. a second modified H5 HA antigen bound to the second binding site; and
b. measuring the formed bridging complex.

2. The method of claim 1, wherein step (a) comprises contacting the antibody with the first and/or second modified H5 HA antigens in a solution that contains a soluble H1 HA construct.

3. The method of claim 2, wherein the amount of the H1 HA construct is sufficient to reduce the formation of bridging complexes comprising antibodies that are cross-reactive with H1 and H5.

4. The method of claim 2 or claim 3, wherein the H1 HA construct comprises the full ectodomain of an H1 HA.

5. The method of any one of claims 2 to 4, wherein the H1 HA construct comprises the head domain of an H1 HA.

6. The method of any one of claims 1 to 5, wherein the first and second modified H5 HAs comprise a first and a second detectable label, respectively, and measuring the formed bridging complex comprises generating a signal that is dependent on the proximity of the first and second detectable labels.

7. The method of any one of claims 1 to 5, wherein the first modified H5 HA antigen comprises a first detectable label and the second modified H5 HA antigen is immobilized on a solid phase support, and
wherein step (b) comprises measuring the first detectable label on the solid support.

8. The method of any one of claims 1 to 5, wherein:
the first modified H5 HA antigen comprises a first detectable label,
the second modified H5 HA antigen comprises a binding moiety, and
the step of measuring the formed bridging complex comprises binding the binding moiety to a binding partner of the binding moiety on a solid phase support, and measuring the first detectable label on said solid support.

9. The method of any one of claims 1-8, wherein the step of forming the bridging complex is carried out in solution, and the bridging complex is subsequently immobilized on the solid support by the binding of the binding moiety to the binding partner.

10. The method of any one of claims 7 to 9, wherein the detectable label on the solid support undergoes a wash step to remove free detectable label in solution, prior to measuring the detectable label.

11. The method of any one of claims 1 to 10, wherein the first and second modified H5 HAs antigens do not include HA domains involved in HA trimerization.

12. The method of any one of claims 1 to 11, wherein the HA amino acid sequences comprised in the modified H5 HAs are amino acids from a head domain.

13. The method of any one of claims 1 to 10, wherein the HA amino acid sequences comprised in the modified H5 HAs are amino acids from an HA1 HA fragment.

14. The method of any one of claims 1 to 13, further comprising analyzing one or more calibration standards with known H5 antibody activity values, generating a calibration equation based on the measurements from the calibration standards, and applying the calibration equation to convert the detected level of the bridging complex in the sample into an H5 antibody activity value.

15. The method of any one of claims 1 to 14, wherein the sample is selected from the group consisting of blood, plasma, serum, milk, and a combination thereof.

16. The method of any one of claims 1 to 15, wherein the sample is obtained from a mammal or a bird.

17. The method of claim 16, wherein the mammal is a human or a cow.

18. The method of claim 16, wherein the sample is obtained from a non-human animal.

19. A method of detecting an antibody to a first antigen comprising
contacting a sample with a first antigen of the antibody linked a detectable label and second antigen of the antibody linked to a binding moiety under conditions suitable for the antibody to bind to the first and second antigens and form a bridging complex comprised thereof,
optionally contacting the sample with an unlabeled blocking antigen that is not the same as the first and second antigens; and
detecting the detectable label.

20. The method of claim 19, comprising contacting the second antigen with a binding partner that binds the binding moiety.

21. The method of claim 20, wherein the binding partner is immobilized on a surface.

22. The method of any one of claims 19-21, wherein the first and second antigens are selected from peptides, proteins, carbohydrates, lipids, and combinations thereof.

23. The method of any one of claims 19-22, wherein the first and second antigens are the same.

24. The method of any one of claims 19-23, wherein the blocking antigen is selected to reduce or prevent binding of cross-reactive antibodies that recognize epitopes shared between the first and second antigens and the blocking antigen; optionally wherein the blocking antigen reduces the formation of bridging complexes formed by cross-reactive antibodies.

25. The method of any one of claims 19-24, wherein the detectable label is selected from the group consisting of fluorescent dyes, enzymes, electrochemiluminescent labels, chemiluminescent labels, biotin, affinity tags, and a combination thereof.

26. The method of any one of claims 19-25, wherein the detectable label is selected from the group consisting of fluorescein, Alexa Fluor dyes, Cy dyes, horseradish peroxidase (HRP), alkaline phosphatase (AP), acridinium esters, luminol, FLAG, His-tag, HA-tag, and a combination thereof.

27. The method of any one of claims 19-26, wherein the binding partners are selected from the group consisting of biotin and avidin, biotin and streptavidin, biotin and neutravidin, hapten and anti-hapten antibody pairs, digoxigenin and anti-digoxigenin antibodies, fluorescein and anti-fluorescein antibodies, and complementary oligonucleotides.

28. The method of any one of claims 19-27, wherein detection comprises measuring the level of the detectable label.

29. The method of any one of claims 19-28, further comprising one or more washing steps.

30. The method of any one of claims 19-29, wherein some antibodies in the sample are cross-reactive to the first, second, and blocking antigens.

31. The method of any one of claims 19-30, wherein the first and second antigens are an H5N1 HA protein or a fragment thereof and/or the blocking antigen is an H1N1 HA protein or a fragment thereof.

32. The method of any one of claims 19-31, wherein the sample is from a subject.

33. The method of claim 32, wherein the subject is selected from the group consisting of a human, a domestic animal, a livestock animal, a companion animal, a wild bird, a poultry animal, and combinations thereof.

34. A method of determining if a subject has been exposed to a first antigen comprising
detecting an antibody the binds to the first antigen in a sample from the subject according to the method of any one of claims 19-33, and
determining that the subject has been exposed to the first antigen when the detectable label is detected.

35. The method of claim 34, wherein determining that the subject has been exposed to the first antigen when the detectable label is detected at a level that exceeds a threshold level.

36. The method of claim 35, wherein the threshold level if higher than the level detected in a control sample or average of control samples analyzed in the same manner as the sample from the subject.

37. The method of claims 35 and 36, wherein the threshold level is a predetermined standard.

38. A method of diagnosing a subject with a disease or disorder associated with exposure a first antigen comprising determining the subject has been exposed to the first antigen according to the method of any one of claims 34-37.

39. The method of claim 38, further comprising treating the subject for the disease or disorder.

40. A method of determining if a subject has been vaccinated against the first antigen comprising determining the subject has been exposed to the first antigen according to the method of any one of claims 34-39.

41. A composition, kit, method, or reagent as described herein including, but not limited to, the text, drawings, or combination thereof.

42. The method of any one of claims 2-5, wherein the H1 HA construct comprises a mixture of two or more H1 HA proteins or fragments thereof.

43. The method of claim 6, wherein the first modified H5 HA antigen comprises a first detectable label or the second modified H5 HA antigen comprises a second detectable label,
optionally wherein the first detectable label and the second detectable label are different; and
wherein measuring the formed bridging complex comprises generating a signal that is dependent on the proximity of the first detectable label and the second detectable label.

44. The method of claim 43, wherein the first detectable label and/or the second detectable label is selected from electrochemiluminescent labels, fluorescent labels, chemiluminescent labels, enzymatic labels, affinity labels, and combinations thereof.

45. The method of claim 43 or claim 48, wherein the first detectable label and/or the second detectable label is selected from fluorescein, Alexa Fluor dyes, Cy dyes, horseradish peroxidase (HRP), alkaline phosphatase (AP), acridinium esters, luminol, FLAG, His-tag, HA-tag, SULFO-TAG, and combinations thereof.

46. The method of claim 7, wherein the first detectable label is selected from the group consisting of an electrochemiluminescent label, a fluorescent label, a chemiluminescent label, an enzymatic label, an affinity label, and combinations thereof.

47. The method of claim 7 or claim 46, wherein the first detectable label is selected from the group consisting of fluorescein, Alexa Fluor 488, Alexa Fluor 647, Cy3, Cy5, horseradish peroxidase (HRP), rhodamine, alkaline phosphatase (AP), β-galactosidase, acridinium esters, Luciferase, luminol, FLAG, His-tag, HA-tag, SULFO-TAG, and combinations thereof.

48. The method of claim 8, wherein the binding moiety is selected from biotin, affinity tags, haptens, and combinations thereof.

49. The method of claim 48, wherein the binding partner comprises streptavidin, avidin, or neutravidin immobilized on the solid phase support.

50. The method of claim 8, wherein the binding moiety comprises biotin and the binding partner comprises streptavidin.

51. The method of claim 1, wherein the first and second modified H5 HA antigens comprise one or more amino acid sequences selected from SEQ ID NO:1, SEQ ID NO:2, SEQ ID NO:3, SEQ ID NO:7, SEQ ID NO:8, SEQ ID NO:9, SEQ ID NO:10, and SEQ ID NO:12 or one or more amino acid sequences having at least 70%, at least 80%, at least 85%, at least 90%, at least 95%, at least 98%, at least 99%, such as from about 70% to about 99%, from about 70% to about 95%, from about 70% to about 90%, from about 70% to about 85%, from about 70% to about 80%, from about 70% to about 75% sequence identity to SEQ ID NO:1, SEQ ID NO:2, SEQ ID NO:3, SEQ ID NO:4, SEQ ID NO:7, SEQ ID NO:8, SEQ ID NO:9, SEQ ID NO:10, or SEQ ID NO: 12.

52. The method of claim 12, wherein the first and second modified H5 HA antigens comprise one or more amino acid sequences from the head domain of an H5 HA (H5 HA amino acid sequences).

53. The method of claim 52, wherein the H5 HA amino acid sequence is SEQ ID NO:12 or an amino acid sequence having at least 70%, at least 80%, at least 85%, at least 90%, at least 95%, at least 98%, at least 99%, such as from about 70% to about 99%, from about 70% to about 95%, from about 70% to about 90%, from about 70% to about 85%, from about 70% to about 80%, from about 70% to about 75% sequence identity to SEQ ID NO:12.

54. The method of claim 13, wherein the H1N1 HA antigen has an amino acid sequence selected from SEQ ID NO:5, SEQ ID NO:6, SEQ ID NO:15, SEQ ID NO:17, and SEQ ID NO:18, or an amino acid sequence having at least 70%, at least 80%, at least 85%, at least 90%, at least 95%, at least 98%, at least 99%, such as from about 70% to about 99%, from about 70% to about 95%, from about 70% to about 90%, from about 70% to about 85%, from about 70% to about 80%, from about 70% to about 75% sequence identity to SEQ ID NO:5, SEQ ID NO:6, SEQ ID NO:15, SEQ ID NO:17, and SEQ ID NO:18.

55. The method of claim 54, wherein the H1N1 HA antigen has the amino acid sequence SEQ ID NO:18 or an amino acid sequence having at least 70%, at least 80%, at least 85%, at least 90%, at least 95%, at least 98%, at least 99%, such as from about 70% to about 99%, from about 70% to about 95%, from about 70% to about 90%, from about 70% to about 85%, from about 70% to about 80%, from about 70% to about 75% sequence identity to SEQ ID NO:18.

56. A method of determining if a subject has been exposed to H5N1 comprising measuring the formed bridging complex in a sample obtained from the subject.

57. The method of claim 56, wherein determining that the subject has been exposed to the first antigen when the detectable label is detected at a level that exceeds a threshold level.

58. The method of claim 57, wherein the threshold level if higher than the level detected in a control sample or average of control samples analyzed in the same manner as the sample from the subject.

59. The method of claim 57 or claim 58, wherein the threshold level is a predetermined standard.

60. A method of diagnosing a subject with an infection caused by H5N1 comprising determining the subject has been exposed to H5N1 according to the method of any one of claims 56-59.


SEQUENCE LISTING (18 sequences)

SEQ ID NO: 1 - protein, 568 aa, unidentified
MEKIVLLLATVSLVKSDQICIGYHANNSTEQVDTIMEKNVTVTHAQDILERTHNGKLCDLNGVKPLILRDCSVAGWLLGNPMCDEFINVPEWSYIVEKASPANDLCYPGNFNDYEELKHLLSRINHFEKIQIIPKSSWSNHDASSGVSSACPYLGRSSFFRNVVWLIKKNSTYPTIKRSYNNTNQEDLLVLWGIHHPNDAAEQTKLYQNPTTYISVGTSTLNQRLVPEIATRPKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNTPQRERRRKKRGLFGAIAGFIEGGWQGMVDGWYGYHHSNEQGSGYAADQESTQKAIDGVTNKVNSIINKMNTQFEAVGREFNNLERRIENLNKKMEDGFLDVWTYNTELLVLMENERTLDFHDSNVKNLYDKVRLQLRDNAKELGNGCFEFYHKCDNECMESVKNGTYDYPQYSEEARLNREEISGVKLESMGTYQILSIYSTVASSLALAIMVAGLSLWMCSNGSLQCRICI

SEQ ID NO: 2 - protein, 568 aa, unidentified
MEKIVLLLAIVSLVKSDQICIGYHANNSTEQVDTIMEKNVTVTHAQDILEKTHNGKLCDLNGVKPLILRDCSVAGWLLGNPMCDEFINVPEWSYIVEKASPANDLCYPGDFNDYEELKHLLSRTNHFEKIQIIPKSSWSNHDASSGVSSACPYHGRSSFFRNVVWLIKKNSAYPTIKRSYNNTNQEDLLVLWGIHHPNDAAEQTKLYQNPTTYISVGTSTLNQRLVPEIATRPKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNTPQRERRRKKRGLFGAIAGFIEGGWQGMVDGWYGYHHSNEQGSGYAADKESTQKAIDGVTNKVNSIIDKMNTQFEAVGREFNNLERRIENLNKQMEDGFLDVWTYNAELLVLMENERTLDFHDSNVKNLYDKVRLQLRDNAKELGNGCFEFYHKCDNECMESVKNGTYDYPQYSEEARLNREEISGVKLESMGTYQILSIYSTVASSLALAIMVAGLSLWMCSNGSLQCRICI

SEQ ID NO: 3 - protein, 568 aa, unidentified
MEKIVLLLAIVSLVKSDQICIGYHANNSTEQVDTIMEKNVTVTHAQDILEKTHNGKLCDLNGVKPLILRDCSVAGWLLGNPMCDEFTNVPEWSYIVEKASPANDLCYPGDFNDYEELKHLLSRINHFEKIQIIPKSSWSNHDASSPVSSACPYHGRSSFFRNVVWLIKKNSTYPTIKRSYNNTNQEDLLVLWGIHHPNDAAEQTKLYQNPTTYISVGTSTLNQRLVPEIATRPKVNGQSGRMEFFWTILKPIDAINFESNGNFIAPEYAYKIVKERDSAIMKSELEYGNCNTKCQTPMGAINSSMPFHNIHPLTIGECPKYVKSNRLVLATGLRNTPQRERRRKKRGLFGAIAGFIEGGWQGMVDGWYGYHHSNEQGSGYAADKESTQKAIDGVTNKVNSIIDKMNTQFEAVGREFNNLERRIESLNKKMEDGFLDVWTYNAELLVLMENERTLDFHDSNVKNLYDKVRLQLRDNAKELGNGCFEFYHKCDNECMESVKNGTYDYPQYSEEARLNREEISGVKLESMGTYQILSIYSTVCELPSTGNHGSLVLSLWMSNGSLQCRICI

SEQ ID NO: 4 - protein, 531 aa, synthetic construct
MEKIVLLLAIVSLVKSDQICIGYHANNSTEQVDTIMEKNVTVTHAQDILEKTHNGKLCDLNGVKPLILRDCSVAGWLLGNPMCDEFTNVPEWSYIVEKASPANDLCYPGDFNDYEELKHLLSRINHFEKIQIIPKSSWSNHDASSPVSSACPYHGRSSFFRNVVWLIKKNSTYPTIKRSYNNTNQEDLLVLWGIHHPNDAAEQTKLYQNPTTYISVGTSTLNQRLVPEIATRPKVNGQSGRMEFFWTILKPIDAINFESNGNFIAPEYAYKIVKERDSAIMKSELEYGNCNTKCQTPMGAINSSMPFHNIHPLTIGECPKYVKSNRLVLATGLRNTPQRERRRKKRGLFGAIAGFIEGGWQGMVDGWYGYHHSNEQGSGYAADKESTQKAIDGVTNKVNSIIDKMNTQFEAVGREFNNLERRIESLNKKMEDGFLDVWTYNAELLVLMENERTLDFHDSNVKNLYDKVRLQLRDNAKELGNGCFEFYHKCDNECMESVKNGTYDYPQYSEEARLNREEISGVKLESMGTYQ

SEQ ID NO: 5 - protein, 566 aa, unidentified
MEAKLLVLFCTFAALKADTICIGYHANNSTDTVDTVLEKNVTVTHSVNLLENSHNGKLCSLNGIAPLQLGKCNVAGWLLGNLECDLLLTANSWSYIIETSNSENGTCYPGEFIDYEELREQLSSVSSFEKFEIFPKASSWPNHETTKGVTAACSYLGASSFYRNLLWMTKKGTSYPKLSKSYTNNKGKEVLVLWGVHHPPTTSEQQTLYQNVDAYVSVGSSKYNRRFTPEIAARPKVRGQAGKMNYYWTLLDQGDTITFEATGNLIAPWYAFALNKGSDSGIITSDAPVHNCDTKCQTPYGALNSSLPFQNVHPITIGECPKYVKSTKLRMATGLRNVPSIQSRGLFGAIAGFIEGGWTGMIDGWYGYHHQNEQGSGYAADQKSTQSAIDGITNKVNSVIEKMNTQFTAVGKEFNNLERRIENLNKKVDDGFLDVWTYNAELLVLLENERTLDFHDSNVRNLYEKVKSQLRNNAKEIGNGCFEFYHKCDDECMESVKNGTYDYPKYSEESKLNREEIDGVKLESMGVYQILAIYSTVASSLVLLVSLGAVSFWMCSNGSLQCRICI

SEQ ID NO: 6 - protein, 566 aa, unidentified
MEAKLFVLFCAFTTLEADTICVGYHANNSTDTVDTILEKNVTVTHSVNLLENSHNGKLCSLNGVAPLQLGKCNVAGWILGNPECDLLLTANSWSYIIETSDSENGTCYPGEFIDYEELREQLSSVSSFERFEIFPKANSWPNHETTKGITAACSYSGTLSFYRNLLWIVKRGNSYPKLSKSYTNNKGKEVLIIWGVHHPPTTSDQQSLYQNADAYVSVGSSKYNRRFTPEIAARPKVKGQAGRMNYYWTLLDQGDTITFEATGNLIAPWYAFALNKGSGSGIITSDTPVHNCDTKCQTPHGALNSSLPFQNVHPITIGECPKYVKSTKLRMATGLRNVPSIQSRGLFGAIAGFIEGGWTGMIDGWYGYHHQNEQGSGYAADQKSTQIAIDGISNKVNSVIEKMNTQFTAVGKEFNDLEKRIENLNKKVDDGFLDVWTYNAELLVLLENERTLDFHDFNVRNLYEKVKSQLRNNAKEIGNGCFEFYHKCDDECMESVKNGTYNYPKYSEESKLNREEIDGVKLESMEVYQILAIYSTVASSLVLLVSLGAISFWMCSNGSLQCRICI

SEQ ID NO: 7 - protein, 567 aa, unidentified
MENIVLLLAIVSLVKSDQICIGYHANNSTEQVDTIMEKNVTVTHAQDILEKTHNGKLCDLNGVKPLILKDCSVAGWLLGNPMCDEFIRVPEWSYIVERANPANDLCYPGSLNDYEELKHLLSRINHFEKILIIPKSSWPNHETSLGVSAACPYQGAPSFFRNVVWLIKKNDTYPTIKISYNNTNREDLLILWGIHHSNNAEEQTNLYKNPTTYISVGTSTLNQRLVPKIATRSQVNGQRGRMDFFWTILKPDDAIHFESNGNFIAPEYAYKIVKKGDSTIMKSGVEYGHCNTKCQTPVGAINSSMPFHNIHPLTIGECPKYVKSNKLVLATGLRNSPLREKRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYNKVRLQLRDNAKELGNGCFEFYHKCDNECMESVRNGTYDYPQYSEEARLKREEISGVKLESIGTYQILSIYSTAASSLALAIMMAGLSLWMCSNGSLQCRICI

SEQ ID NO: 8 - protein, 567 aa, unidentified
MENIVLLLAIVSLVKSDQICIGYHANNSTEQVDTIMEKNVTVTHAQDILEKTHNGKLCDLNGVKPLILKDCSVAGWLLGNPMCDEFIRVPEWSYIVERANPANDLCYPGSLNDYEELKHMLSRINHFEKIQIIPKSSWPNHETSLGVSAACPYQGAPSFFRNVVWLIKKNDAYPTIKISYNNTNREDLLILWGIHHSNNAEEQTNLYKNPITYISVGTSTLNQRLAPKIATRSQVNGQRGRMDFFWTILKPDDAIHFESNGNFIAPEYAYKIVKKGDSTIMKSGVEYGHCNTKCQTPVGAINSSMPFHNIHPLTIGECPKYVKSNKLVLATGLRNSPLREKRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESVGTYQILSIYSTAASSLALAIMMAGLSLWMCSNGSLQCRICI

SEQ ID NO: 9 - protein, 567 aa, synthetic construct
MKKIVLLFATISLVKSDHICIGYHANNSTEKVDTIMEKNVTVTQAQDILEKTHNGKLCDLNGVKPLILKDCSVAGWLLGNPLCDEFTNVPEWSYIVEKANPVNDLCYPGSFNDYEELKHLLSRINHFEKIQIIPKNSWSDHEASLGVSATCSYQGNSSFFRNVVWLIKKNNAYPTIKKDYNNTNREDLLILWGIHHPNDEAEQTRLYQNPTTYISIGTSTLNQRLVPRIATRPKINGQSGRIDFFWTILKPNDAIHFESNGNFIAPEYAYKIIKKGDSTIMRSEVKYGNCNTRCQTPIGAINSSMPFHNIHPLTIGECPKYVKSSKLVLATGLRNSPQKERRRKRGLFGAIAGFIEGGWQGMVDGWYGYHHSNEQGSGYAADKESTQRAIDGVTNKVNSIIDKMNTQFEAVGREFNNLERRIENLNKKMEDGFLDVWTYNAELLVLMENERTLDFHDSNVRNLYDKVRLQLKDNAKELGNGCFEFYHKCNNECMESVRNGTYDYPQYSEEARLKREEISGVKLESMGIYQILSIYSTVASSLVLAIMMAGLSLWMCSNGSLQCRICI

SEQ ID NO: 10 - protein, 568 aa, unidentified
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRESRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLESIGTYQILSIYSTVASSLALAIMMAGLSLWMCSNGSLQCRICI

SEQ ID NO: 11 - protein, 8 aa, synthetic construct
DYKDDDDK

SEQ ID NO: 12 - protein, 286 aa, unidentified
LEKTHNGKLCDLNGVKPLILKDCSVAGWLLGNPMCDEFIRVPEWSYIVERANPANDLCYPGSLNDYEELKHLLSRINHFEKILIIPKSSWPNHETSLGVSAACPYQGAPSFFRNVVWLIKKNDTYPTIKISYNNTNREDLLILWGIHHSNNAEEQTNLYKNPTTYISVGTSTLNQRLVPKIATRSQVNGQRGRMDFFWTILKPDDAIHFESNGNFIAPEYAYKIVKKGDSTIMKSGVEYGHCNTKCQTPVGAINSSMPFHNIHPLTIGECPKYVKSNKGSHHHHHH

SEQ ID NO: 13 - protein, 10 aa, synthetic construct
ILKKATAYIL

SEQ ID NO: 14 - protein, 10 aa, synthetic construct
EQKLISEEDL

SEQ ID NO: 15 - protein, 565 aa, unidentified
MKVKLLVLLCTFTATYADTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGNQKALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 16 - skipped

SEQ ID NO: 17 - protein, 566 aa, synthetic construct
MKAILVVLLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDKGVTAACPHAGAKSFYKNLIWLVKKGNSYPKLNQTYINDKGKEVLVLWGIHHPPTIAAQESLYQNADAYVFVGTSRYSKKFKPEIATRPKVRDQEGRMNYYWTLVEPGDKITFEATGNLVVPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 18 - protein, 286 aa, synthetic construct; identical to SEQ ID NO: 12